(19) United States
(12) Patent Application Publication
(10) Pub. No.: US 2016/0136080 A1
(43) Pub. Date: May 19, 2016

(54) Colloidally Stable Dispersions Based on Modified Galactomannans

(71) Applicant: LUBRIZOL ADVANCED MATERIALS, INC., Cleveland, OH (US)

(72) Inventors: Ashoke K. Sengupta, Grover, MO (US); Eric H. Anderson, Cleveland, OH (US)

(21) Appl. No.: 14/897,329

(22) PCT Filed: Jun. 17, 2014
(86) PCT No.: PCT/US2014/042734

§ 371 Date: Dec. 10, 2015

Related U.S. Application Data
(60) Provisional application No. 61/836,303, filed on Jun. 18, 2013.

(51) Int. Cl. A61K 8/73; A61Q 19/00; A61K 8/04; C08B 37/00

(52) U.S. Cl. CPC A61K 8/73; C08B 37/0087; A61Q 19/00; A61K 8/04; A61K 2800/48

(57) ABSTRACT

The disclosed technology relates to a multi-functional additive for personal-care and cosmetic product applications, more particularly, a naturally derived multifunctional additive for use in skin-care formulations, which provides steric stabilization of pigment particles and/or emulsion droplets in aqueous compositions, good suspension-stability (i.e., against settling/creaming) of the compositions, and increased utilization of particulate, skin-care benefit agents contained in the compositions.

### BACKGROUND OF THE TECHNOLOGY

The disclosed technology relates to aqueous dispersion compositions for personal-care and cosmetic product formulations, comprising a multifunctional additive, more particularly, a naturally-derived multifunctional additive for use in skin-care formulations, which provides steric stabilization of pigment particles and/or emulsion droplets in aqueous compositions, good suspension-stability (i.e., against settling/creaming) of the said dispersion compositions, and increased utilization of particulate, skin-care benefit agents contained in the compositions.

A wide variety of personal-care and cosmetic products are intended to be stable dispersions. These stable dispersions may include “colloidally-stable” oil droplets and/or solid pigment particles, which must remain suspended in a continuous phase, such as, for example, an aqueous solution. By “colloidally-stable” it is meant the dispersion is stable against flocculation (i.e., agglomeration of suspended pigment particles or emulsion droplets) and coalescence (i.e., fusion of two or more emulsion droplets). Such dispersions can be in the form of oil-in-water (O-W) emulsions, or a complex O-W emulsion containing both emulsion droplets and pigment particles as separate dispersed phases. The dispersed phase components may comprise various skin-care actives or benefit agents such as, for example, inorganic sunscreen actives, e.g., zinc oxide (ZnO), titanium dioxide (TiO₂), organic (oil-like) sunscreen actives, e.g., octyl methoxycinnamate, color pigments, e.g., iron oxides, TiO₂, anti-wrinkle/blemish (soft-focus) pigment, e.g., alumina, and/or sensory-boosting pigment, such as silica or talc, to name a few.

Particles suspended in a liquid have an innate tendency to flocculate and coalesce (as with emulsion droplets) in order to minimize their surface area (and thus, their surface free energy) unless rendered colloidally-stable against agglomeration and coalescence. The greater the colloidal stability of the suspended particles, the greater the ability to achieve a larger total exposed surface area of the particles for a given dosage, and hence a greater benefit derived from the particulate benefit agents. Accordingly, maximizing the colloidal stability of skin-care dispersions having particulate, skin-care benefit agents contained in the dispersed phase(s) would imply maximizing the skin-care benefits for a given dosage of the benefit agents. Alternatively, maximizing colloidal stability would enable minimizing the dosage of benefit agents in attaining a targeted level of benefit.

Furthermore, for suspended particles of high-density (e.g., inorganic pigments) and low-density (e.g., oils) materials, and/or of a relatively large size, settling or creaming occurs rapidly, unless the viscosity (e.g., low-shear-rate viscosity) of the continuous phase is sufficiently high to minimize the rate of gravity-driven settling/creaming. Settling/creaming of suspended particles would impair the shelf-life and aesthetics of dispersion-based products. Since agglomeration of suspended particles leads to an increase in particle size, an additional benefit of maximizing the colloidal stability of dispersions, would be in providing an aid to minimize the rate of settling and creaming.

Stabilizing suspended particles against agglomeration and coalescence would require: i) strong inter-particle repulsion; and ii) preventing the particles from closely approaching one another, while the particles undergo their random Brownian motion. A highly effective way of providing for a strong particle-to-particle repulsion is to coat the particles with a linear polymer, which would require that the polymer has a strong affinity for the particle surface. Yet another requirement, albeit somewhat opposed to the one above, is that, upon coating the particles, the polymer must flare out sufficiently into the continuous phase in a loop-and-tail fashion to impart what is known as inter-particle “steric repulsion.” A further requirement for a linear polymer to function as a steric-stabilizer, which is rooted in the mechanistic elements of inter-particle steric repulsion, is that the polymer dosage must be less than the critical overlap concentration of the polymer, C. The critical overlap concentration is the concentration at which polymer chains in solution start to interpenetrate each other. The higher the weight average molecular weight (“Mw”) of a polymer, the lower is its C, for which very high molecular weight polymers (e.g., M_{w}>>1 million Dalton) are not effective as steric stabilizers. On the other hand, an essential step for inter-particle agglomeration is the Brownian collision of suspended particles, wherein the particles closely approach one another due to their random Brownian motion, which ultimately leads to their agglomeration if the inter-particle repulsion is not sufficiently strong to prevent agglomeration. Accordingly, a way to hinder inter-particle agglomeration is to retard the rate of Brownian collision of suspended particles by thickening the continuous phase of dispersions. As noted earlier, increased viscosity (low-shear-rate viscosity) of the continuous phase also minimizes settling and creaming of the suspended particles.

The high-performing dispersants or emulsifiers, i.e. the additives used in producing highly stable dispersions, as well as the highly effective thickening agents known in the art are generally polymeric. Even though polymers are the best suited additives to function as dispersant, emulsifier, and thickener, it is generally difficult to have a single polymer or even mixtures of polymers serving all of these functionalities adequately at the same time. For example, hydrophobically-modified, water-soluble/dispersible polymers can be effective emulsifiers for O-W emulsions, but they generally cannot function as pigment-dispersants for hydrophilic pigments such as ZnO, TiO₂, alumina, silica, iron oxides and so forth. Similarly, if structurally suited, hydrophilic polymers can be highly effective thickening agents, and dispersants for hydrophilic pigments, but not emulsifiers for O-W emulsions unless they are hydrophobically-modified. Also, since thermodynamic incompatibility of polymers is a norm rather than an exception, (i.e., the phenomenon of phase-separation of polymers when two or more non-interacting polymers are brought into a mixture) stabilizing a mixture of different types of particles (pigments, emulsion droplets) in complex emulsions by using a mixture of an emulsifier-polymer, a dispersant-polymer, and a thickening agent-polymer, is generally not a viable option.

Compounding the difficulties mentioned above, in recent years, consumers increasingly gravitate towards natural ingredient-based skin-care products due to an increased awareness of the deleterious effects of certain synthetic ingredients. However, natural ingredients often present performance-limitations and poor sensory properties.

Galactomannan polymers are a class of naturally-derived hydrophilic polymer, which in recent years have drawn interest for use in cosmetic compositions as a naturally-derived thickening agent. However, as a thickening agent, they are generally found to be much less efficient than synthetic polymers such as, for example, cross-linked acrylate polymers (Carbomer polymers), and a highly effective natural polymer such as xanthan gum. In that vein, galactomannan polymers typically fall much short of Carbomer polymers and xanthan gum in providing for a sufficiently high (e.g., 0.5 rpm, Brookfield viscosity of 25,000-50,000 cps) low-shear-rate viscosity required for good stability against settling and creaming, even when used at much higher dosages (e.g., 1 wt. %), as compared to typical dosages (e.g., 0.1-0.5 wt. %) with Carbomer polymers and xanthan gum. Furthermore, most galactomannan polymers, unless subjected to chemical reactions to become hydrophobically-modified, tend to show a relatively poor interfacial activity, meaning that their ability to adsorb or bind onto an oil-water interface is unacceptably weak. However, the first and foremost requirement for a molecule to work as an effective emulsifier is that it must have a strong ability to adsorb at an oil-water interface, and any extensive chemical modification of a natural polymer for increasing its interfacial-activity would imply a vast detraction from being naturally-derived. Furthermore, according to Wang and Somasundaran (J. Colloid Interface Sci., 2007), galactomannan polymers, such as guar gum and locust bean gum adsorb onto inorganic pigments (e.g., talc) due to hydrogen (H)-bonding, but upon adsorption, take on a flat rather than an expanded (loops and tails) conformation, due to a strong H-bonding interaction with the pigment surface. For all of the foregoing reasons, one skilled in the art would not expect the prospects of employing galactomannan type polymers to efficiently provide for de-agglomeration of pigment particles, stable (i.e., against agglomeration and coalescence) emulsification of oil droplets, or stable (i.e., against settling and creaming) suspension of pigment particles and oil droplets in aqueous dispersions. Equally significant for the scope and significance of the present technology is that, the use of galactomannan polymers at relatively high dosages is not a viable option for personal-care and cosmetic products, unlike, albeit within limits, for industrial and food products. The reason is that polymers tend to show poor sensory and texture properties which are not acceptable for personal-care and cosmetic products, when used at relatively high levels such as, for example, 1.5 wt. % or higher.

The object of the present technology is to provide compositions for skin-care formulations by providing a multifunctional additive composed of naturally-derived ingredients, wherein the additive is capable of enabling: steric stabilization of pigment particles and emulsion droplets in aqueous compositions, good suspension-stability (i.e., against settling/creaming) of the compositions, and increased utilization of particulate skin-care benefit agents contained in the compositions. The additive necessarily contains, as a key component, a naturally-derived, water-soluble or water-dispersible polymer that is not subjected to a chemical reaction for any hydrophobic modification of the polymer via covalent-bonding.

A related object is having the said additive produced in a liquid—or a gel or any other wetted-solid-form, with the said product-form being necessarily such that it enables wetting of the natural polymer solids prior to the incorporation of the additive into skin-care formulations.

### SUMMARY OF THE TECHNOLOGY

As discussed above, there are three basic properties a polymeric dispersing agent must include to function efficiently (i.e., being effective at a relatively low dosage), for example in aqueous dispersion compositions. The polymeric dispersing agent must 1) have a relatively strong affinity for the surface of the oil droplets or pigment particles to be dispersed, and thus be able to adsorb or bind onto the droplet- or the particle-surface, 2) be able to flare out sufficiently into the continuous phase in a loop-and-tail fashion to impart inter-particle steric repulsion, and 3) be able to be dosed at a concentration less than its critical overlap concentrations C*. Likewise, for complex dispersion systems containing a combination of both pigment particles and oil droplets, a single polymeric dispersing agent would be advantageous to avoid thermodynamic incompatibility of polymers in polymer-mixtures.

The inventors have discovered that galactomannan compounds can be formulated to meet each of the properties of an effective polymeric dispersant for aqueous dispersions, and further, that a single galactomannan can be employed to disperse complex dispersion systems including a combination of solid particles and enhanced oil droplets.

Thus, in one aspect the disclosed technology provides a multi-functional additive suitable for preparing a colloidally stable aqueous dispersion. The multifunctional additive can include (a) a substrate having at least one hydrogen bonding site and (b) a water-soluble or water-dispersible galactomannan polymer having at least one hydrogen bonding site. The substrate can be at least one of: (i) a solid particle, wherein said at least one hydrogen bonding site is on said solid particle surface, or (ii) an amphiphillic anchoring agent. The amphiphillic anchoring agent can have (A) an oil soluble fatty tail, and (B) a polar portion that provides the at least one hydrogen bonding site. The ratio (“R_{H/Mw}”) between the number of hydrogen bonding sites, “H,” on the polar portion (B) to the weight average molecular weight, Mw, of the amphiphillic anchoring agent can be between about 1.65×10⁻³ and 0.2. The amphiphillic anchoring agent can also have an anchoring agent efficiency factor (AAEF) of 1.65×10⁻³ or greater.

In another embodiment, the ratio of galactose to mannose in the water-soluble or water-dispersible galactomannan polymer can be between about 1:5 to 1:8, or more particularly, can be cassia tora gum. In some embodiments the cassia tora gum can be glycerol substituted. Where the cassia tora gum is substituted by reacting it, for example, with chloroglycerine, the glycerol molar substitution can be from 0.25:1 to 3:1 glycerol to cassia tora gum.

In some embodiments, the weight ratio of the galactomannan to the amphiphillic anchoring agent can be 0.1:1 or greater.

In another embodiment, the multifunctional additive can additionally include a thickening agent. In some embodiments, the thickening agent can be included in a synergistic amount.

In one embodiment the multi-functional additive can be in the form of a dry powder, with a residual content of volatiles (e.g., water, alcohol, etc.) of less than 30% by weight.

In another embodiment the multifunctional additive can include a hydrophilic medium and/or a hydrophobic medium. In some embodiments, the multifunctional additive can include both a hydrophilic medium and a hydrophobic medium.

In one embodiment, the multifunctional additive can be in the form of an emulsion of the hydrophobic medium in the hydrophilic medium, such as an oil-in-water (O-W) emulsion. In another embodiment, the multi-functional additive can be in the form of an inverse emulsion of the hydrophilic medium in the hydrophobic medium.

Also disclosed herein is a personal care or cosmetically acceptable composition comprising the multifunctional additive. More particularly, the personal care or cosmetically acceptable composition can include (a) a substrate having at least one hydrogen bonding site, (b) a water-soluble or water-dispersible galactomannan polymer having at least one hydrogen bonding site, (c) a personal care or cosmetically acceptable aqueous medium, and (d) a personal care or cosmetically acceptable hydrophobic medium. The galactomannan can be bonded to the substrate via a noncovalent bond, such as a hydrogen bond.

The substrate can include at least one of (i) a solid particle, wherein said at least one hydrogen bonding site is on a surface of said solid particle, (ii) an amphiphillic anchoring agent, said amphiphillic anchoring agent being comprised of (A) an oil soluble fatty tail, and (B) a polar portion that provides said at least one hydrogen bonding site, wherein the ratio (“R_{H/Mw}”) between the number of hydrogen bonding sites, “H,” on the polar portion (B) to the weight average molecular weight, Mw, of the amphiphillic anchoring agent is between about 1.65×10⁻³ and 0.2, or (iii) both (i) and (ii).

Also disclosed is a method of providing a colloidally stable dispersion of a hydrophobic medium in a continuous hydrophilic medium. The method can comprise including in the dispersion an amphiphillic anchoring agent and a galactomannan. Further disclosed is a method of providing a colloidally stable dispersion of a solid particle together with a hydrophobic medium in a continuous hydrophilic medium. The method can comprise including in the dispersion an amphiphillic anchoring agent and a galactomannan. Yet another disclosure is a method of providing a colloidally stable dispersion of a solid particle in a continuous hydrophilic medium. The method can comprise including in the dispersion a galactomannan.

### DETAILED DESCRIPTION OF THE TECHNOLOGY

Various preferred features and embodiments will be described below by way of non-limiting illustration.

The general goal of the disclosed technology is to provide colloidally stable dispersions. Often the desired colloidally stable dispersion is a complex dispersion. By “complex” dispersion it is meant that the dispersion contains both liquid and solid particulates. For example, a complex dispersion can include an emulsion of a hydrophobic medium in a continuous hydrophilic medium, along with a suspension of solid particles in the same continuous hydrophilic medium. Where the term “complex” is not expressly noted herein, it is to be understood that any dispersion generically discussed can be complex dispersions.

As used herein, the term “hydrophilic medium” encompasses aqueous media. Hydrophilic media include, but are not limited to, for example, water, glycerin, alcohols, including glycols and polyols, and combinations thereof. Preferably the hydrophilic medium is water or glycerin. Water may be deionized, industrial soft water, or any suitable grade of water.

The hydrophobic medium is not particularly limited. The hydrophobic medium can be any personal care or cosmetically acceptable natural or synthetic oil medium or mixtures thereof, or natural or synthetic waxes suitable for use in personal care or cosmetic applications.

The oils may be volatile or non-volatile, or a mixture of both. Exemplary personal care and cosmetically acceptable oil mediums include, for example, vegetable oils, animal oils, hydrocarbon oils, fatty alcohols, fatty acid esters, silicone oils, oily UV absorbers and sunscreens, fragrance oils, and mixtures thereof.

Exemplary vegetable oils include, but are not limited to apricot stone, avocado oil, macadamia nut oil, olive oil, coconut oil, jojoba oil, corn oil, sunflower oil, palm oil, soybean oil, castor oil, peanut oil, walnut oil, rapeseed oil, almond oil, palm oil, coconut oil palm kernel oil, groundnut oil, wheat germ oil, cottonseed oil, lucerne oil, poppy oil, pumpkin oil, primrose oil, millet oil, barley oil, rye oil, wheat germ oil, safflower oil, candlenut oil, passiflora oil, hazelnut oil, shea butter, calophyllum oil, sysymbrium oil, and mixtures thereof.

Synthetically modified vegetable oils (mono-, di-, and triglycerides) derived through the esterification of glycerol, a monoglyceride, or a diglyceride with a fatty acid(s) also is suitable as the oil phase component. They are prepared by techniques well known in the art, or by glycerolysis of animal fats and vegetable oils in the presence of a base at elevated temperature and under an inert atmosphere (See RSC Green Chemistry Book Series, The Royal Society of Chemistry, The Future of Glycerol: New Uses Of A Versatile Material, Chapter 7, Mario Pagliaro and Michele Rossi, © 2008). Fatty acids suitable for use in the esterification reaction include saturated and unsaturated C₈-C₃₀ fatty acids.

Exemplary animal oils include, but are not limited to, neatsfoot oil, liquid fractions of beef tallow, lanolin, lanolin derivatives (e.g., isopropyl lanolate, isocetyl lanolate), tallow, mink oil, cholesterol, fish oil, sperm whale oil, and mixtures thereof.

Exemplary hydrocarbon oils include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils typically contain about 12 to 19 carbon atoms. Branched chain hydrocarbon oils, including hydrocarbon polymers, typically will contain more than 19 carbon atoms. Specific non-limiting examples of these hydrocarbon oils include paraffin oil, mineral oil, petrolatum, liquid polyolefin oils, fluorinated and perfluorinated oils, saturated and unsaturated dodecane, isohexadecane, isododecane, saturated and unsaturated tridecane, saturated and unsaturated tetradecane, saturated and unsaturated pentadecane, saturated and unsaturated hexadecane, polybutene, polydecene, and mixtures thereof. Branchedchain isomers of these compounds, as well as of higher chain length hydrocarbons, can also be used, examples of which include highly branched, saturated or unsaturated, alkanes such as the permethyl substituted isomers, e.g., the permethyl-substituted isomers of hexadecane and eicosane, such as 2,2,4,4,6,6,8,8-octamethyl-10-methylundecane and 2,2,4,4,6,6-hexamethyl-8-methylnonane, available from Permethyl Corporation. Hydrocarbon polymers such as polybutene and polydecene are also useful. Mineral oils and petrolatums include cosmetic, USP and NF grades and are commercially available from Penreco under the Drakeol® and Penreco® trade names.

The liquid polyolefin oils are typically poly(α-olefins) that have been hydrogenated. Polyolefins for use herein can be prepared by the polymerization of C₄ to about C₁₄ olefinic monomers. Non-limiting examples of olefinic monomers for use in preparing the polyolefin liquids herein include ethylene, propylene, 1-butene, 1-pentene, 1-hexene, 1-octene, 1-decene, 1-dodecene, 1-tetradecene, and 1-hexadecene, branched isomers such as isobutylene, 4-methyl-1-pentene, and mixtures thereof. In one aspect, a suitable hydrogenated polyolefin is the copolymer of isobutylene and butene. A commercially available material of this type is Panalane® L-14E (INCI Name: Hydrogenated Polyisobutene) marketed by Lipo Chemicals Inc, Patterson, N.J.

Fluorinated oils include perfluoropolyethers described in European Patent No. EP 0 486 135 and the fluorohydrocarbon compounds described in International Patent Application Publication No. WO 93/11103. The fluoridated oils may also be fluorocarbons such as fluoramines, e.g., perfluorotributylamine, fluorinated hydrocarbons, such as perfluorodecahydronaphthalene, fluoroesters, and fluoroethers.

The fatty alcohols suitable for use in the compositions of the technology include, but are not limited to, the saturated and unsaturated C₈-C₃₀ fatty alcohols. Exemplary fatty alcohols include capryl alcohol, pelargonic alcohol, capric alcohol, decyl alcohol, undecyl alcohol, lauryl alcohol, myristyl alcohol, cetyl alcohol, isocetyl alcohol, stearyl alcohol, isostearyl alcohol, cetearyl alcohol, palmitoleyl alcohol, elaidyl alcohol, sterol, oleyl alcohol, linoleyl alcohol, elaidolinoleyl alcohol, linolenyl alcohol, ricinoleyl alcohol, arachidyl alcohol, icocenyl alcohol, behenyl alcohol, erucyl alcohol, lignoceryl alcohol, ceryl alcohol, montanyl alcohol, myricyl alcohol, and mixtures thereof. Fatty alcohols are widely available and can be obtained through the hydrogenation of esterified vegetable and animal oils and fats.

Suitable fatty acid esters are the mono-, di- or triesters of linear and/or branched mono-, di- or tricarboxylic acids having 2 to 44 carbon atoms with linear and/or branched saturated or unsaturated alcohols having 1 to 22 carbon atoms.

Monoesters suitable as oil components are, for example, the methyl esters and isopropyl esters of fatty acids having 12 to 22 carbon atoms, for example, methyl laurate, methyl stearate, methyl cocoate, methyl oleate, methyl erucate, isopropyl palmitate, isopropyl myristate, isopropyl stearate, isopropyl isostearate, or isopropyl oleate.

Other suitable monoesters are, for example, butyl stearate, hexyl laurate, isohexyl laurate, isodecyl neopentanoate, isooctyl stearate, lauryl lactate, isostearyl lactate, cetearyl octanoate isononyl palmitate, isononyl isononanoate, 2-ethylhexyl laurate, octyl stearate, decyl stearate, cetyl stearate, stearyl stearate, oleyl stearate, 2-hexyldecyl stearate, 2-ethylhexyl palmitate, cetyl palmitate, 2-octyldodecyl palmitate, myristyl myristate, oleyl myristate, decyl oleate, isodecyl oleate, oleyl oleate, oleyl erucate, or erucyl oleate, and esters which are obtainable from technical grade aliphatic alcohol cuts and technical grade, aliphatic carboxylic acid mixtures, e.g., esters of unsaturated fatty alcohols having 12 to 22 carbon atoms and saturated and unsaturated fatty acids having 12 to 22 carbon atoms, as are obtainable from animal and vegetable fats.

Diesters suitable as oil components are, for example, those prepared from dicarboxylic acids (e.g., oxalic, malonic, succinic, glutaric, adipic, pimelic, suberic, azelaic, sebacic, phthalic, isophthalic, terephthalic, maleic, glutaconic and traumatic acids) and linear and/or branched saturated or unsaturated alcohols having 1 to 22 carbon atoms. Examples of dicarboxylic acid esters include, but are not limited to, diisopropyl adipate, dibutyl adipate, dioctyl adipate, di(2-ethylhexyl) adipate, dibutyl sebacate or di(2-hexyldecyl) succinate, diisotridecyl acelate.

Diol esters prepared from a diol (e.g., glycols, polyglycols, and linear or branched diols) and a C₆ to C₂₂ linear or branched, saturated or unsaturated monocarboxylic acid can be employed as the oil component. Exemplary diol esters are, for example, ethylene glycol dioleate, ethylene glycol diisotridecanoate, propylene glycol di(2-ethylhexanoate), polypropylene glycol monooleate, polypropylene glycol monostearate, butanediol diisostearate and neopentyl glycol dicaprylate.

Triesters suitable as the oil component are, for example, those prepared from tricarboxylic acids (e.g., citric, isocitric, aconitic, carballylic, trimesic, trimellitic acid) and linear and/or branched saturated or unsaturated alcohols having 1 to 22 carbon atoms. Exemplary triesters include but are not limited to, trimethyl citrate, triethyl citrate, tristearyl citrate, triisopropyl citrate, triisostearyl citrate, trioctyldodecyl citrate, trioleyl citrate. triisodecyl citrate, triisopropyl citrate, tributyl citrate, tris(2-ethylhexyl)citrate, trioctyl trimellitate, and mixtures thereof.

Also suitable are the esterification products of aliphatic, difunctional alcohols having 2 to 36 carbon atoms with monofunctional saturated and unsaturated C₈ to C₃₀ fatty acids. Other polyhydric alcohol esters include the partial esters of polyglycerols. These esters contain 2 to 10 glycerol units and are esterified with 1 to 4 saturated or unsaturated, optionally hydroxylated C₈ to C₃₀ fatty acids. Representative partial esters of polyglycerols include, but are not limited to, diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate, decaglycerol trihydroxystearate, and mixtures thereof.

Suitable silicone oils include, but are not limited to, polydimethylsiloxanes, methylphenylpolysiloxanes, silicones modified by amines, silicones modified by alcohols and fatty acids, cyclic polysiloxanes, and mixtures thereof. They can be volatile or non-volatile.

Silicone oils include polyalkyl, polyaryl siloxanes, or polyalkylaryl siloxanes which conform to the following formula:

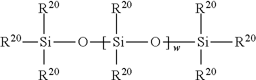

wherein R²⁰ is an aliphatic group, independently selected from alkyl, alkenyl, and aryl, R²⁰ can be substituted or unsubstituted, and w is an integer from 1 to about 8,000. Suitable unsubstituted R²⁰ groups for use in the present technology include, but are not limited to alkoxy, aryloxy, alkaryl, arylalkyl, arylalkenyl, alkamino, and ether-substituted, hydroxyl substituted, and halogen substituted aliphatic and aryl groups.

In one aspect of the technology, exemplary R²⁰ alkyl and alkenyl substituents include C₁-C₅ alkyl and C₁-C₅ alkenyl groups. In another aspect, R²⁰ is methyl. Exemplary aryl groups in the foregoing embodiments include phenyl and benzyl moieties.

Exemplary siloxanes are polydimethylsiloxane, polydiethylsiloxane, and polymethylphenylsiloxane. These siloxanes are available, for example, from Momentive Performance Materials in their Viscasil R and SF 96 series, and from Dow Corning marketed under the Dow Corning 200 series. Exemplary polyalkylaryl siloxane fluids that may be used include, for example, polymethylphenylsiloxanes. These siloxanes are available, for example, from Momentive Performance Materials as SF 1075 methyl phenyl fluid or from Dow Corning as 556 Cosmetic Grade Fluid, or from Wacker Chemical Corporation, Adrian, Mich., under the trade name Wacker-Belsil® PDM series of phenyl modified silicones (e.g., PDM 20, PDM 350 and PDM 1000).

Cyclic polysiloxanes (cyclomethicones) can be represented by the formula:

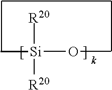

wherein the substituent R²⁰ is as defined above, and the number of repeat units, k, ranges from about 3 to about 7 in one aspect, and from 3 to 5 in another aspect. Additionally, R²⁰ and k can be selected so that the material is volitile or non-volatile. Aryl containing substituents include those which contain alicyclic and heterocyclic five and six member aryl rings and those which contain fused five or six member rings. The aryl rings can be substituted or unsubstituted. Substituents include aliphatic substituents, and can also include alkoxy substituents, acyl substituents, ketones, halogens (e.g., Cl and Br), amines, etc. Exemplary aryl containing groups include substituted and unsubstituted arenes, such as phenyl, and phenyl derivatives such as phenyls with C₁-C₅ alkyl or alkenyl substituents, e.g., allylphenyl, methyl phenyl and ethyl phenyl, vinyl phenyls such as styrenyl, and phenyl alkynes (e.g., phenyl C₂-C₄ alkynes). Heterocyclic aryl groups include substituents derived from furan, imidazole, pyrrole, pyridine, etc. Fused aryl ring substituents include, for example, naphthalene, coumarin, and purine.

Exemplary cyclomethicones are D4 cyclomethicone (octamethylcyclotetrasiloxane), D5 cyclomethicone (decamethylcyclopentasiloxane), D6 cyclomethicone (dodecamethylcyclohexasiloxane), and blends thereof (e.g., D4/D5 and D5/D6). Cyclomethicone and cyclomethicone blends are commercially available from Momentive Performance Materials Inc. as SF1202, SF 1214, SF1256, and SF1258, Dow Corning, Midland, Mich. under the Xiameter® cyclomethicone fluid product designations PMX-0244, PMX-245, PMX-246, PMX-345, and Dow Corning® 1401 fluid.

Suitable fragrance oils include extracts from natural raw materials, such as, essential oils, concretes, absolutes, resins, resinoids, balsams, and tinctures; hydrocarbons, such as, for example, 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane; aliphatic alcohols; cyclic alcohols; cycloaliphatic alcohols; aliphatic ketones; acyclic terpene alcohols; cyclic terpene alcohols; cyclic terpene aldehydes and ketones; and mixtures thereof. Additional fragrance oils are disclosed in U.S. Pat. No. 7,335,631, which is hereby incorporated by reference. Other suitable fragrance oils are disclosed below as fragrances and perfumes.

Waxes include those derived from plant, animal/insect, mineral, petroleum and synthetic sources. Synthetically modified natural (plant and animal/insect) waxes are also contemplated herein. Exemplary plant derived waxes include but are not limited to bayberry wax, candelilla wax, hydrolyzed candelilla wax, carnauba wax, ethoxylated carnauba wax (e.g., PEG-12 carnauba wax), hydrolyzed carnauba wax, carnauba acid wax, hydrogenated castor wax, esparto wax, hydrogenated Japan wax, hydrogenated jojoba oil, jojoba oil esters, sulfurized jojoba oil, ouricury wax, palm kernel wax, and hydrogenated rice bran wax. Exemplary animal/insect derived waxes include but are not limited to beeswax, oxidized beeswax, ethoxylated beeswax (e.g., PEG-6 beeswax, PEG-8 beeswax, PEG-12 beeswax, PEG-20 beeswax), dimethicone copolyol beeswax esters and dimethiconol beeswax ester (e.g. Bis-Hydroxyethoxypropyl Dimethicone Beeswax Esters, Dimethicone PEG-8 Beeswax, and Dimethiconol Beeswax available from Noveon, Inc. under the Ultrabee™ trademark), Chinese wax, shellac wax, spermaceti wax, mink wax, and lanolin wax. Exemplary mineral waxes include but are not limited to ceresin waxes, montan wax, montan acid wax, and ozokerite. Exemplary petroleum waxes include paraffin waxes, microcrystalline waxes, and oxidized microcrystalline waxes. Exemplary synthetic waxes include synthetic beeswax, synthetic candelilla wax, synthetic carnauba wax, synthetic Japan wax, synthetic jojoba oil, polyolefin waxes (e.g., polyethylene wax), ethylene glycol diesters or triesters of fatty acids containing 18 to 40 carbon atoms. Mixtures of two or more of the forgoing waxes and classes of waxes are also contemplated.

In one aspect the disclosed technology provides a multi-functional additive suitable for preparing a colloidally stable dispersion in combination with a hydrophilic medium and hydrophobic medium. The multifunctional additive can include (a) a substrate having at least one hydrogen bonding site and (b) a water-soluble galactomannan polymer having at least one hydrogen bonding site.

The substrate of the multi-functional additive can be at least one of: (i) a solid particle, wherein said at least one hydrogen bonding site is on a surface of said solid particle, or (ii) an amphiphillic anchoring agent.

Solid particle substrates suitable for use in the multi-functional additive will contain at least one hydrogen bonding site at the particle surface. Solid particles can include hydrophilic particles and hydrophilically modified particles. Examples of solid particles contemplated for use in the composition include, but are not limited to, solid particulate fragrances and perfumes, botanicals, inorganic pigments (e.g., inorganic oxides, silicates, carbonates, sulfates, including those of alkaline earth metals) exfoliants, antidandruff agents, insoluble materials (clay, laponite, microsponges, cosmetic beads and flakes), opacifiers and pearlizing agents, humectants, emollients, antioxidants, deodorizing agents, pH adjusting agents, buffer agents, chelation agents, viscosity modifiers, structuring agents, deposition aids, UV protection agents, sunscreens, insect repellants, antiperspirants, pharmaceutical and cosmetic actives, skin and hair conditioners, preservatives, and electrolytes. However, the solid particle is not particularly limited and may be any hydrophilic or hydrophilically modified solid particle having at least one hydrogen bonding site that is desired to be dispersed in an aqueous composition.

The solid particle can be present in the multi-functional additive in a range from about 0.1 to about 70 wt. %, from about 0.5 to about 65 wt. %, from about 1 to about 60 wt. %, from about 1.5 to 50 wt. %, from about 2 to 30 wt. %, from about 2.5 to about 15 wt. %, and from about 5 to about 10 wt. %, based on the total weight of the multi-functional additive.

Amphiphillic anchoring agent substrates suitable for use in the multi-functional additive can have (A) at least one oil soluble fatty tail (i.e., oil soluble portion), and (B) a polar (i.e., hydrophilic) portion having at least one hydrogen bonding site.

The oil soluble portion of the amphiphillic anchoring agent can be an alkyl, aryl or acyl group comprised of from 4 to 40 carbon atoms, or 5 to 36 carbon atoms, or even 6 to 34 or 7 to 28 carbon atoms. The oil soluble portion can be saturated or unsaturated (mono- or polyunsaturated). While the amphiphillic agent will have at least one oil soluble portion, it can also have two or more, for example three, oil soluble portions. The oil soluble portion may contain a hydrogen bonding group, for example if the tail is prepared from an acid, such as 12-hydroxystearic acid. However, the hydrogen bonding groups on the tail do not count toward the hydrogen bonding groups on the polar portion of the amphiphillic anchoring agent.

The polar portion of the amphiphillic anchoring agent can be anionic, cationic, zwitterionic, or nonionic. While the amphiphillic agent will have at least one polar portion, it can also have two or more, for example three, polar portions. The hydrogen bonding group on the polar portion can be a hydrogen bond acceptor, such as an oxygen group, or a hydrogen bond donor, such as hydrogen.

In one embodiment, the amphiphillic anchoring agent can dually function as an amphiphillic anchoring agent and an emulsifying agent.

The amphiphillic anchoring agent can be a solid or a liquid. Examples of suitable amphiphillic anchoring agents include amphiphillic fatty acids, amphiphillic fatty esters, amphiphillic fatty alcohols, amphiphillic phospholipids, amphiphillic sterols and amphiphillic polymers.

In one embodiment, suitable amphiphillic anchoring agents are those in which the ratio between the number of hydrogen bonding sites, “H,” on the polar portion (B) to the weight average molecular weight, Mw, of the amphiphillic anchoring agent, herein referred to as “R_{H/Mw},” is between about 1.65×10⁻³ and 0.2.

When in a mixed media containing a hydrophobic medium and a hydrophilic medium, the amphiphillic anchoring agent functions to provide bonding points for the water-soluble or water-dispersible galactomannan polymer to adsorb or bind onto the hydrophobic or oil soluble medium. As such, it is desirable that the oil soluble portion (A) of the amphiphillic anchoring agent be solubilized in the hydrophobic medium and the polar portion having the at least one hydrogen bonding site (B) rests at or near the interface between the hydrophobic medium and hydrophilic medium. In such a way, at least one hydrogen bonding site on the water-soluble/water-dispersible galactomannan can encounter and bind to the at least one hydrogen bonding site on the polar portion of the anchoring agent.

The efficiency of an amphiphillic anchoring agent compound in increasing the adsorption of a galactomannan at an oil-water interface relies on two factors:
i) the ability of the amphiphillic anchoring agent to adsorb at an oil-water interface, and
ii) its ability to bond with the galactomannan through hydrogen-bonding.
   Suitable amphiphillic anchoring agents are those having an anchoring agent efficiency factor (AAEF) of 1.65×10⁻³ or greater, or 1.7×10⁻³ or greater or 1.75 or 1.8×10⁻³ or greater, up to about 0.01, 0.05, 0.1, or 0.2. The AAEF is the calculated efficiency of an amphiphilic anchoring-agent to increase the adsorption of a galactomannan onto an oil droplet in an oil-in-water (OW) emulsion, based on the number of hydrogen-bonding groups present in the polar portion of the anchoring agent compound per mole of the compound in proportion to a solvation factor (SF). A high SF value would signify that the anchoring-agent compound has a strong affinity for either a hydrophobic medium or hydrophilic medium, depending on whether the anchoring agent is more oil-soluble or water-soluble, respectively. The stronger the affinity of an amphiphilic anchoring agent for a hydrophobic medium or hydrophilic medium in which it is soluble, the weaker would be the prospects for any hydrogen-bonded complex of the galactomannan and amphiphilic anchoring agent to adsorb at the oil-water interface of oil droplets in O-W emulsions. The AAEF value for the amphiphilic anchoring agent compounds is given by:

AAEF=R_{H/Mw}/SF

where:
SF is equal to (1+Absolute value of (10−HLB))
   wherein HLB denotes the hydrophilic-lipophilic balance value of the amphiphillic anchoring agent, which can be determined per the Griffin method, which is known in the art, or which can be readily referenced by those ordinarily skilled in the art. Note that compounds having an HLB value of less than 10 tend to be oil-soluble, whereas compounds having an HLB value of greater than 10 tend to be water-soluble. Compounds having an HLB value of 0 are lipophilic (i.e., not amphiphillic) and compounds having an HLB value of 20 are hydrophilic (i.e., not amphiphillic). Thus, amphiphillic anchoring agents having an HLB of from about 0.1 to about 19.9, or 0.25 to about 19.75, or 0.5 to about 19.5 can be employed in the multi-functional additive.

Table II shows the calculated AAEF values of exemplary amphiphillic anchoring agent compounds.

TABLE II

| | Number of | | | AAEF, |
|---|---|---|---|---|
| Amphiphilic | Hydrogen- | | | Anchoring- |
| Anchoring | bonding Groups | | | agent |
| Agent | in the Polar | Molecular | HLB | Efficiency |
| Compound | Portion | Weight | Value | Factor |
| Glyceryl Stearate | 6 | 358.56 | 3.8 | 0.002324 |
| Sorbitan Palmitate | 9 | 402.57 | 6.7 | 0.005199 |
| Sorbitan Oleate | 9 | 428.6 | 4.3 | 0.003134 |
| Sorbitan Stearate | 9 | 430.62 | 4.7 | 0.003317 |
| Methyl Glucose | 10 | 1238.77 | 6.6 | 0.001835 |
| Sesquistearate | | | | |
| Sucrose Cocoate | 20 | 342 | 15 | 0.009747 |
| Lauryl Glucoside | 10 | 348.47 | 13 | 0.007174 |
| Glycol Stearate | 4 | 328.53 | 2.9 | 0.001503 |

The amphiphillic anchoring agent can be present in the multi-functional additive in a range from about 0.1 to about 75 wt. %, from about 0.5 to about 65 wt. %, from about 1 to about 50 wt. %, from about 2 to 30 wt. %, from about 2.5 to about 15 wt. %, and from about 5 to about 10 wt. %, based on the total weight of the multi-functional additive.

The multi-functional additive also includes at least one water-soluble or water-dispersible galactomannan polymer, which acts as a dispersing agent. Galactomannans (used interchangeably with the term polygalactomannan herein) are a class of polysaccharides that are found in the endosperm material of seeds from leguminous plants such as Cyamopsis tetragonoloba (guar gum), Cesalpinia spinosa (tara gum), Ceratonia siliqua (locust bean gum), and other members of the Leguminosae family. The structure of galactomannan can be illustrated by a typical structure of a galactomannan obtained from the endosperm of cassia seed:

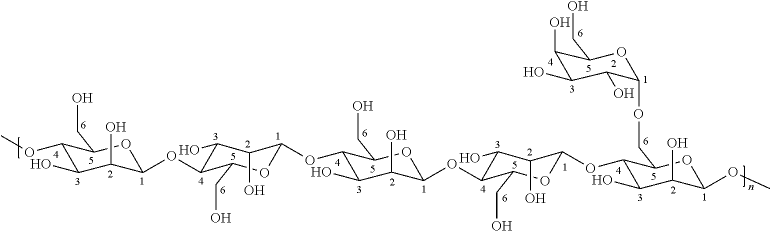

wherein n is an integer representing the number of repeat units in the polymer. Due to the hydroxyl and ether oxygen groups on galactomannan, galactomannan has a strong affinity for the hydrogen bonding sites on the substrate.

As can be seen in the structure above, galactomannan is composed of a backbone of 1→4-linked β-D-mannopyranosyl main chain units (also designated herein as a mannoside unit or residue, or simply mannose) with recurring 1→6-linked α-D-galactosyl side groups (also designated herein as a galactoside unit or residue, or simply galactose) branching from the number 6 carbon atom of a mannopyranose residue in the polymer backbone. The mannoside and galactoside units are generically referred to herein as glycoside units or residues.

The galactomannan polymers of the different Leguminosae species differ from one another in the frequency of the occurrence of the galactoside side units branching from the polymannoside backbone. It is well understood by those skilled in the art that natural galactomannans, even when obtained from a single source, will contain varying ranges of mannose to galactose ratios. Accordingly, these mannose to galactose ratios are reported as average ratios. Some specific galactomannan species are, for example, that derived from guar gum, tara gum, and locust bean gum. The average ratio of D-mannoside to D-galactoside contained in guar gum is approximately 1.5 or 2:1, approximately 3:1 for tara gum, and approximately 4:1 for locust bean gum. In general, the average ratio of mannose to galactose in the galactomannan must be sufficient to allow the galactomannan to loop out into the continuous medium to provide steric repulsion between dispersed particles. Accordingly, the preferred average ratio, referred to herein as “M:G ratio”, of D-mannoside to D-galactoside units in the galactomannan suitable to employ as a dispersing agent can be from about 5:1 to about 49:1 in one embodiment, from about 6:1 to about 35:1 in another embodiment, from about 7:1 to about 25:1 in still another embodiment, from about 8:1 to about 10:1 in a further embodiment. An important source of polygalactomannan suitable to act as a dispersing agent in the multi-functional additive is isolated from the endosperm of the seeds of Cassia tora and Cassia obtusifolia (collectively known as cassia gum). The average ratio of D-mannosyl to D-galactosyl units in the polygalactomannan contained in cassia gum (derived from Cassia tora and Cassia obtusifolia) is at least 5:1 and up to about 8:1. In a preferred embodiment, the Cassia gum encompassing the M:G ratios set forth above is obtained from the endosperm of Cassia tora, Cassia obtusifolia, and/or mixtures thereof. The monosaccharide content of Cassia gum can be determined using a method adapted from Englyst et al. (“Determination of Dietary Fibre as Non-Starch Polysaccharides by Gas-Liquid Chromatography.” Analyst (117), November 1992, pp. 1707-1714).

Galactomannans are hydrocolloids that have a high affinity for water due to the pendant alcohol groups, which act as hydrogen bonding sites. However, when used in their natural form galactomannans have suffered some drawbacks from a water solubility standpoint. An unsubstituted polymannose backbone is completely insoluble in water. The attachment of galactose side units to the C-6 atom in the recurring mannose residues of the polymannose backbone increases the water solubility of the polymer, particularly in cold water (i.e., ambient temperature and below). The greater the galactose side unit substitution, the greater is the cold water solubility properties of the polygalactomannan. Consequently, lower ratios of D-mannosyl to D-galactosyl units in the polygalactomannan leads to better cold water solubility. For example, the polygalactomannan contained in guar gum (average D-mannosyl to D-galactosyl ratio 2:1) is mostly soluble in cold water, while the polygalactomannan obtained from cassia gum (average D-mannosyl to D-galactosyl ratio of at least 5:1) is only sparingly soluble in cold and hot water.

For galactomannan having higher average ratios of D-mannosyl to D-galactosyl, such as that derived from cassia tora gum, the solubility of the galactomannan in water can be enhanced by modifying the galactomannan. For example, the galactomannan can be nonionically, anionically, cationically, and/or amphoterically derivatized. Such chemical modification of the galactomannan can lead to incorporation of nonionic, anionic, cationic, and amphoteric moieties, and combinations thereof onto the backbone, which leads to various physical properties changes. For instance, derivatized cassia gums exhibit cold water or improved cold water solubility.

Modification of galactomannan to improve physical properties can occur via molecular substitution. As used herein and throughout the specification, the terms molecularly substituted and molecular substitution mean appending a substituent selected from nonionic, anionic, cationic, and amphoteric containing moieties, as well as combinations thereof, to the C-6 carbon atom of the galactose side unit and/or to the C-6 carbon atom of the mannose repeating backbone units of the polygalactomannan, for instance a cassia derived polygalactomanan. Functionalization reagents containing these moieties are co-reacted with the hydroxyl group that is bonded to the C-6 carbon atom of the galactose and mannose residues that make up the polygalactomannan. In other words, a hydroxyl hydrogen is replaced by a moiety derived from the functionalization reagent. In one embodiment, the hydroxyl hydrogen on the C-6 carbon atom is replaced by a moiety derived from the functionalization reagent. The galactomannans suitable for use herein can be derivitized, for example as taught in columns 5 and 6 in U.S. Pat. No. 7,262,157B2 issued Aug. 28, 2007.

Representative nonionic groups include but are not limited to hydroxymethyl, hydroxyethyl, hydroxypropyl, and hydroxybutyl, glycerol. Representative anionic groups include but are not limited to carboxymethyl, carboxyethyl, and carboxypropyl. Cationic groups can include quaternary ammonium groups. The amphoteric substituents can be selected from any radical or residue that contains both a positive and negative charge. Representative amphoteric substituents include betaine, amino acid, dipeptides, tripeptide and polypeptide residues.

The polygalactomannan used in the practice of the disclosed technology typically has an Mw within the range of 200,000 to 5,000,000 Daltons. In many cases, the polygalactomannan has a weight average molecular weight within the range of 300,000 to 2,000,000 Daltons. It is common for the galactomannan used in the practice of the disclosed technology to have a weight average molecular weight within the range of 400,000 to 1,500,000 Daltons, and even 500,000 to 800,000 Daltons. It is also common for the galactomannan used in this technology to have a number average molecular weight (Mn) which is within the range of 100,000 to 1,500,000 Daltons. In many cases, the polygalactomannan has a number average molecular weight, which is within the range of 200,000 to 1,000,000 Daltons. It is common for the polygalactomannan used in the practice of the disclosed technology to have a number average molecular weight, which is within the range of 300,000 to 900,000 Daltons. The molecular weight of the galactomannan can be varied through controlled degradation procedures known in the art. The weight average molecular weights and number average molecular weights referenced herein can be determined by gel permeation chromatography (GPC) with refractive index and low angle light scattering detectors.

Preferably, the galactomannan employed in the multi-functional additive is derived from cassia tora gum having an average ratio of D-mannosyl to D-galactosyl of greater than about 5:1, and generally from about 5:1 to about 8:1. In a preferred embodiment, the cassia derived galactomannan is molecularly substituted with glycerol, for example by reacting the cassia derived galactomannan with chloroglycerine. The glycerol molar substitution can be from about 0.25:1 to about 3:1, from about 0.5:1 to about 1.5:1 glycerol to cassia tora gum, or from about 0.6:1 to about 1.4:1, or even about 0.7:1 to about 1.3:1. Most preferably the glycerol molar substitution can be about 1 to 1 glycerol to cassia tora gum.

While the galactomannan can be hydrophilically modified, the galactomannan is not hydrophobically modified. For example, the galactomannan is a non-alkylated galactomannan.

The galactomannan can be present in the multi-functional additive in the range of 0.1 to about 75 wt. %, from about 0.5 to about 65 wt. %, from about 1 to about 50 wt. %, from about 2 to 30 wt. %, from about 2.5 to about 15 wt. %, and from about 5 to about 10 wt. %, based on the total weight of the multi-functional additive.

In some embodiments the level of galactomannan in the multi-functional additive can depend upon the level of amphiphillic anchoring agent present in the additive, and to which phase (i.e., water-phase or oil-phase) the galactomannan and/or the amphiphilic anchoring agent are added. For example, where the water-soluble/water-dispersible galactomannan and the amphiphilic anchoring agent are added to the water phase, the weight ratio of galactomannan, such as glyceryl cassia gum, to amphiphillic anchoring agent can be prepared at a starting ratio of 1:1 galactomannan to amphiphillic anchoring agent or greater, or 1.25:1 or greater, or even 1.5:1 or 2:1 or greater. Another example, where an oil-soluble amphiphillic anchoring agent is first added to the oil phase, the weight ratio of galactomannan, such as glyceryl cassia gum, to amphiphillic anchoring agent can be prepared at a starting ratio of 0.1:1 galactomannan to amphiphillic anchoring agent or greater, or 0.25:1 or greater, 0.5:1 or greater, or even 1:1 or greater. Generally, at a weight ratio of galactomannan to amphiphillic anchoring agent of more than about 10:1 the efficiency of the interaction diminishes.

Without limiting to any specific theory, it is speculated that when the multifunctional additive is added to a hydrophilic medium, such as water in an O-W emulsion, and the weight-ratio of galactomannan, for example glyceryl cassia gum, to amphiphilic anchoring agent is varied, the relative amounts of (a) free (i.e., unbonded) amphiphilic anchoring-agent compound, (b) hydrogen-bonded complex of galactomannan and amphiphillic anchoring agent, and (c) free (unbonded) galactomannan, affect the emulsification performance of the multi-functional additive. More particularly, it is speculated that the relative affinities of (a), (b), and (c) for the oil-phase in the O-W emulsion, which is expected to be more for (a) than for (b) and (c), have implications on the competitive adsorption of these species on the oil-phase of O-W emulsion. Similarly, it is speculated that the amounts of (a), (b), and (c) that are not adsorbed on the oil-phase have implications on the flocculation of emulsion droplets due to a phenomenon known in the art as “depletion flocculation,” which is induced by un-adsorbed polymers and surfactants, including relative efficacies of the foregoing species in inducing depletion flocculation.

In addition to the substrate and the water-soluble galactomannan polymer, the multi-functional additives can optionally include a thickener. Particulate thickening agents employed in the multi-functional additive are necessarily such that they can interact with the galactomannan, for example, glyceryl cassia, through hydrogen-bonding, and if polymeric, are thermodynamically compatible with the galactomannan (e.g., glyceryl cassia).

The thickener can be one that thickens in a hydrophilic medium or in a hydrophobic medium. Thickeners that thicken in the oil phase include, for example, waxes, hydrophobically modified metal oxides, and layered silicates and aluminates such as fumed silica, fumed alumina, smectite clays, and polymers.

The multi-functional additive may further comprise a suspending agent at concentrations effective for suspending water insoluble material in a dispersed form or for modifying the viscosity of the composition.

Thickeners and suspending agents useful herein for a hydrophilic medium include anionic polymers and nonionic polymers. Useful herein are vinyl polymers such as cross linked acrylic acid polymers with the INCI name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, cassia gum, xanthan gum, gum arabic, tragacanth, galactan, carob gum, guar gum, karaya gum, carrageenan, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

In a preferred embodiment the thickening agent can be a particulate thickener, such as, for example, smectite clay, fumed silica, fumed alumina, laponite, and mixtures thereof, or a polymeric thickener, such as, for example, xanthan gum, acrylate cross-polymers, and mixtures thereof.

Commercially available thickeners useful herein include Carbomers available under the trade names Carbopol® 934, Carbopol® 940, Carbopol® 950, Carbopol® 980, and Carbopol® 981 polymers, all available from Lubrizol Advanced Materials, Inc., acrylates/steareth-20 methacrylate copolymer having the trade name Acrysol™ 22 available from Rohm and Haas (Dow Chemical Company), nonoxynyl hydroxyethylcellulose available under the trade name Amercell™ Polymer HM-1500 available from Amerchol (Dow Chemical Company); methylcellulose available under the trade name Benecel®, hydroxyethyl cellulose available under the trade name Natrosol®, hydroxypropyl cellulose available under the trade name Klucel®, cetyl hydroxyethyl cellulose available under the trade name Polysurf® 67, all supplied by Hercules (Ashland Inc.), ethylene oxide and/or propylene oxide based polymers available under the trade names Carbowax® PEGs, Polyox™ resins, and Ucon® Fluids, all supplied by Amerchol (Dow Chemical Company).

The multi-functional additive can be in the form of a powder dispersion or liquid dispersion.

A solid form of the multi-functional additive can comprise at least one solid particle, at least one amphiphillic anchoring agent in dry form, at least one water-soluble galactomannan polymer in dry form, and optional thickeners in dry form. The solid form of the multi-functional additive can also comprise, consist of, or consist essentially of at least one solid particle and the water-soluble galactomannan polymer, or the solid form of the multi-functional additive can comprise, consist of, or consist essentially of at least one amphiphillic anchoring agent and the water-soluble galactomannan polymer. The solid form of the multi-functional additive can also comprise, consist of, or consist essentially of at least one solid particle, the water-soluble galactomannan polymer, and the optional thickener. Similarly, the solid form of the multi-functional additive can comprise, consist of, or consist essentially of at least one amphiphillic anchoring agent, the water-soluble galactomannan polymer, and the optional thickener.

The dry form of the multi-functional additive can have a residual content of volatiles of less than 30% by weight. By volatiles it is meant liquids that can vaporize, such as, for example, water, alcohol, etc. The dry form in some embodiments can have less than 20% or less than 10% residual volatiles. A residual volatile content of less than 5%, 4%, 3%, and even less than 2% or 1% is desirable. Ultimately it is desireable for the dry form of the multi-functional additive to be completely free of residual level of volatiles, but it is recognized that such levels may be impracticle to attain.

In one embodiment a dry blend can be prepared by wet blending the galactomannan with an aqueous solution of a water soluble amphiphillic anchoring agent, followed by drying the blend to form the dry powder.

The solid form of the multi-functional additive can be employed to prepare a colloidally stable dispersion, for example, by simply blending it with the hydrophilic medium, followed by blending the resultant solution with the hydrophobic medium and/or a solid particle or a pigment.

As a liquid dispersion, the multi-functional additive can additionally include a personal care or cosmetically acceptable hydrophilic medium and/or a personal care or cosmetically acceptable hydrophobic medium, along with the substrate(s), water-soluble galactomannan polymer, and optional thickener. Here again, the substrate can comprise one or the other or both of the solid particle and a solid or liquid form of the amphiphillic anchoring agent, or it can consist essentially of, or consist of, one or the other or both of the solid particle or amphiphillic anchoring agent.

The liquid dispersion can be prepared as an emulsion of the hydrophobic medium in the hydrophilic medium, for example, as an O-W emulsion, or as an inverse emulsion of the hydrophilic medium in the hydrophobic medium.

Emulsions of the multi-functional additive can be prepared by emulsification methods known in the art. For example, in one method the at least one water-soluble galactomannan polymer can be dissolved or dispersed in the hydrophilic medium at ambient temperature with stirring. Separately, the amphiphillic anchoring agent can be added to the hydrophobic medium to prepare an enhanced oil droplet. The resultant enhanced oil droplet can then be added to the hydrophilic medium with agitation to provide a dispersed enhanced oil droplet. The combined phases can be sheared using a high shear impeller (e.g., dispersion blade agitators known in the art) and/or a homogenizer (e.g., a rotor stator homogenizer).

In another preparation method, the at least one water-soluble galactomannan polymer can be dissolved or dispersed with stirring at ambient temperature in the hydrophobic medium along with the amphiphillic anchoring agent. The resultant solution or dispersion can then be dispersed in the hydrophilic medium with stirring. The combined phases are sheared using a high shear impeller (e.g., dispersion blade agitators known in the art) and/or a homogenizer (e.g., a rotor stator homogenizer).

Alternatively, the multi-functional additive emulsions can be prepared at elevated temperatures in a so-called hot process. A suitable temperature for the hot process ranges from about 30 to 95° C. in one aspect, from about 40 to 85° C. in another aspect, and from about 45 to about 75° C. in a further aspect.

In producing a multi-functional additive in the form of an emulsion, the water-soluble galactomannan polymer can be added as is or formulated into a pre-formed master batch as a solution or dispersion of the water-soluble galactomannan polymer in the hydrophilic medium.

When the water-soluble galactomannan polymer is added as a master batch solution or dispersion, the amount of galactomannan in the master batch solution or dispersion can range from about 70 to about 80 wt. % in one aspect, from about 50 to about 70 wt. % in another aspect, and from about 30 to about 50 wt. % in yet another aspect, and from about 3 to about 10 wt. % in a further aspect.

Inverse emulsion forms of the multi-functional additive also can be prepared by methods known in the art. For example, the water-soluble or water-dispersible galactomannan is first dissolved or dispersed in a hydrophilic liquid medium, and the resulting solution or dispersion is then emulsified in a hydrophobic liquid or wax medium, using an oil-soluble or oil-dispersible emulsifying agent, and by shearing the hydrophilic liquid mixture in the hydrophobic liquid medium. The said emulsifying agent is preferably mixed into the hydrophobic liquid medium homogeneously, prior to adding to the hydrophobic liquid medium, the solution or dispersion of the galactomannan, for emulsification. The emulsifying agent can be an amphiphilic anchoring agent compound of the present invention or it can be a surface active compound (i.e., surfactant) known in art as being an emulsifying agent for water-in-oil emulsions. Such inverse emulsion form of the multifunctional additive can contain an amphiphilic anchoring agent either in the hydrophilic liquid medium or in the hydrophobic liquid medium.

Emulsion forms of the additive can have a pumpable consistency, wherein the Brookfield viscosity of the emulsions, at a spindle speed of 20 rpm, is 40,000 mPa·s or less in one aspect, 20,000 mPa·s or less in another aspect, 10,000 mPa·s or less in still another aspect, 5,000 mPa·s or less in a further aspect, 1000 mPa·s or less in still a further aspect, 500, 100, 50 mPa·s or less in an additional aspect

The multi-functional additive, whether in solid or liquid form can optionally comprise other solid particulate or liquid benefit agents, such as, for example, fragrances, perfumes, botanicals, particulate materials (e.g., exfoliants, and anti-dandruff agents), insoluble materials, opacifiers and pearlizing agents, humectants, emollients, antioxidants, deodorizing agents, pH adjusting agents, buffers, chelation agents, viscosity modifiers, structuring agents, deposition aids, and topically active compounds such as UV protection agents, sunscreens, insect repellents, antiperspirants, cosmeceuticals, pharmaceuticals, skin and hair conditioners, preservatives, and combinations thereof.

The optional benefit agents can be dissolved or dispersed in either the hydrophilic medium or hydrophobic medium depending on the agent's solubility in either medium. Determining whether an optional component is soluble or not in a particular medium is well within the knowledge of ordinary formulators.

It is to be understood that the materials listed above and below can serve more than one function and that the listing of a material in any particular class is not intended as a limitation for that material, and that characterization of an additive or component as having a specific function does not exclude the additive or component from performing another function.

When utilized, each optional component(s) typically is included in an amount of from about 0.0001 to about 25 wt. % in one aspect, from about 0.01 to 20 wt. % in another aspect, from about 0.1 to about 15 wt. % in a still another aspect, from about 0.5 to about 10 wt. % in a further aspect, and from about 1 to about 5 wt. % in a still further aspect, based on the total weight of the multi-functional additive. The amounts employed will vary with the purpose and character of the product and can be readily determined by one skilled in the formulation arts and from the literature.

### Fragrances and Perfumes

The fragrance and perfume components that may be used in the context of the technology include natural and synthetic fragrances, perfumes, scents, and essences and any other substances which emit a fragrance. As the natural fragrances, there are those of vegetable origin, such as oil extracts from flowers (e.g., lily, lavender, rose, jasmine, neroli, ylang-ylang), stems and leaves (geranium, patchouli, petitgrain, peppermint), fruits (aniseed, coriander, fennel, needle juniper), fruit skin (bergamot, lemon, orange), roots (mace, angelica, celery, cardamom, costus, iris, sweet flag), woods (pine tree, sandalwood, guaiacum wood, cedar, rosewood, cinnamon), herbs and grasses (tarragon, lemongrass, sage, thyme), needles and twigs (spruce, pine, European red pine, stone pine), and resins and balsam (galbanum, elemi, benzoin, myrrh, frankincense, opopanax), and those of animal origin, such as musk, civet, castoreum, ambergris, or the like, and mixtures thereof.

Examples of synthetic fragrances and perfumes are the aromatic esters, ethers, aldehydes, ketones, alcohols, and hydrocarbons including, but are not limited to, benzyl acetate, phenoxyethyl isobutylate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styralyl propionate, and benzyl salicylate; benzylethyl ether; straight chain alkanals having 8 to 18 carbon atoms, citral, citronellal, citronellyloxyaldehyde, cyclamen aldehyde, hydroxycitronellal, lilial, and bougeonal; ionone compounds, α-isomethyl ionone, and methyl cedryl ketone; anethole, citronellol, eugenol, isoeugenol, geraniol, lavandulol, nerolidol, linalool, phenylethyl alcohol, and terpineol, alpha-pinene, terpenes (e.g., limonene), and balsams, and mixtures thereof.

### Botanicals

Suitable botanical agents may include, for example, extracts from Echinacea (e.g., sp. angustifolia, purpurea, pallida), yucca glauca, willow herb, basil leaves, Turkish oregano, carrot root, grapefruit, fennel seed, rosemary, tumeric, thyme, blueberry, bell pepper, blackberry, spirulina, black currant fruit, tea leaves, such as for, example, Chinese tea, black tea (e.g., var. Flowery Orange Pekoe, Golden Flowery Orange Pekoe, Fine Tippy Golden Flowery Orange Pekoe), green tea (e.g., var. Japanese, Green Darjeeling), oolong tea, coffee seed, dandelion root, date palm fruit, gingko leaf, green tea, hawthorn berry, licorice, sage, strawberry, sweet pea, tomato, vanilla fruit, comfrey, arnica, centella asiatica, cornflower, horse chestnut, ivy, magnolia, oat, pansy, skullcap, seabuckthorn, white nettle, and witch hazel. Botanical extracts may also include, for example, chlorogenic acid, glutathione, glycrrhizin, neohesperidin, quercetin, rutin, morin, myricetin, absinthe, and chamomile.

### Particulates

Suitable particulate materials include pigments, exfoliants, and anti-dandruff agents. Exemplary pigments are metal compounds or semi metallic compounds and may be used in ionic, nonionic or oxidized form. The pigments can be in this form either individually or in admixture or as individual mixed oxides or mixtures thereof, including mixtures of mixed oxides and pure oxides. Examples are the titanium oxides (e.g., TiO₂), zinc oxides (e.g., ZnO), aluminum oxides (for example, Al₂O₃), iron oxides (for example, Fe₂O₃), manganese oxides (e.g., MnO), silicon oxides (e.g., SiO₂), silicates, cerium oxides, zirconium oxides (e.g., ZrO₂), barium sulfate (BaSO₄), nylon-12, and mixtures thereof.

Other examples of pigments include thermochromic dyes that change color with temperature, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide nanoparticles, barium oxide, ultramarine blue, bismuth citrate, hydroxyapatite, zirconium silicate, carbon black particles, and the like.

Numerous cosmetically useful particulate exfoliating agents are known in the art, and the selection and amount is determined by the exfoliating effect desired from the use of the composition, as recognized by those skilled in the cosmetic arts. Useful exfoliating agents include, but are not limited to, natural abrasives, inorganic abrasives, synthetic polymers, and the like, and mixtures thereof. Representative exfoliants include, but are not limited to, ground or powdered pumice, stone, zeolites, nut shells (e.g., almond, pecan, walnut, coconut, and the like), nut meals (e.g., almond, and the like), fruit pits (e.g., apricot, avocado, olive, peach, and the like), hulls, seed and kernel (e.g., oat bran, corn meal, rice bran, grape seed, kiwi seed, wheat, jojoba seed, loofah seed, rose hip seed, and the like), plant matter (e.g., tea tree leaves, corn cob, fruit fibers, seaweed, loofah sponge, microcrystalline cellulose, and the like), bivalve shells (oyster shell, and the like), calcium carbonate, dicalcium pyrophosphate, chalk, silica, kaolin clay, silicic acid, aluminum oxide, stannic oxide, sea salt (e.g., Dead Sea salt), talc, sugars (e.g., table, brown, and the like), polyethylene, polystyrene, microcrystalline polyamides (nylons), microcrystalline polyesters, polycarbonates, and stainless steel fibers. The foregoing exfoliants can be used in the form of granules, powders, flours, and fibers.

Suitable anti-dandruff agents that can be employed in the compositions of the present technology include, but are not limited to, sulfur, zinc pyrithione, zinc omadine, miconazole nitrate, selenium sulfide, piroctone olamine, N,N-bis(2-hydroxyethyl)undecenamide, cade oil, pine tar, coal tar, Allium cepa extract Picea abies extract, and Undecyleneth-6, and the like, and mixtures thereof.

### Insoluble Materials

Insoluble materials suitable for use in the present compositions include, but are not limited to, clay, swellable clay, laponite, gas bubbles, liposomes, microsponges, cosmetic beads and flakes. Cosmetic beads, flakes and capsules can be included in a composition for aesthetic appearance or can function as microencapsulants for the delivery of benefit agents to the skin and/or the hair. Exemplary bead components include, but are not limited to, agar beads, alginate beads, jojoba beads, gelatin beads, Styrofoam™ beads, polyacrylate, polymethylmethacrylate (PMMA), polyethylene beads, Unispheres™ and Unipearls™ cosmetic beads (Induchem USA, Inc., New York, N.Y.), Lipocapsule™, Liposphere™, and Lipopearl™ microcapsules (Lipo Technologies Inc., Vandalia, Ohio), and Confetti II™ dermal delivery flakes (United-Guardian, Inc., Hauppauge, N.Y.).

### Opacifiers and Pearlizing Agents

Some formulations are often opacified by deliberately incorporating pearlescent materials therein to achieve a cosmetically attractive pearl like appearance, known as pearlescence. An opacifier often is included in a composition to mask an undesirable aesthetic property, such as to improve the color of a composition that is darkened due to the presence of a particular ingredient, or to mask the presence of particulate matter in the composition. Opacifiers also are included in compositions to improve the aesthetics and consumer acceptance of an otherwise esthetically unpleasing composition. For example, an opacifier can impart a pearlescent appearance to a clear composition, thereby communicating an appearance of creaminess, mildness and body to the consumer. Persons skilled in the art are aware of problems faced by formulators in consistently preparing a stable pearlescent formulation. A detailed discussion is found in the article “Opacifiers and Pearling Agents in Shampoos” by Hunting, Cosmetic and Toiletries, Vol. 96, pages 65-78 (July 1981), incorporated herein by reference.

The opacifying or pearlescent material can be selected from a number of different chemical classes including inorganic compounds, e.g., various aluminum and magnesium salts, and organic compounds, like fatty alcohols, fatty esters and various polymers and copolymers including, but is not limited to, ethylene glycol mono-stearate, ethylene glycol distearate, polyethylene glycol distearate, stearic alcohol, bismuth oxychloride coated mica, mica coated metal oxides (e.g., titanium dioxide, chromium oxide, iron oxides), myristyl myristate, guanine, glitter (polyester or metallic), and mixtures thereof. Other pearlescent materials can be found in U.S. Pat. No. 4,654,207, U.S. Pat. No. 5,019,376, and U.S. Pat. No. 5,384,114, which are herein incorporated by reference. A representative listing of opacifiers/pearlescent materials is found in the CTFA Cosmetic Ingredient Handbook, J. Nikitakis, ed., The Cosmetic, Toiletry and Fragrance Association, Inc., Washington, D. C., 1988, at page 75.

### Humectants

Humectants suitable for use in the composition of the technology include, but are not limited to, glycerol, polyglycerols, sorbitol, propane-1,2-diol, butane-1,2,3-triol, polyethylene glycols, glucose, mannitol, xyliyol, and mixtures thereof.

### Emollients

Emollients may include silicone oils, functionalized silicone oils, hydrocarbon oils, fatty alcohols, fatty alcohol ethers, fatty acids, esters of monobasic and/or dibasic and/or tribasic and/or polybasic carboxylic acids with mono and polyhydric alcohols, polyoxyethylenes, polyoxypropylenes, mixtures of polyoxyethylene and polyoxypropylene ethers of fatty alcohols, and mixtures thereof. The emollients may be either saturated or unsaturated, have an aliphatic character and be straight or branched chained or contain alicyclic or aromatic rings.

### Antioxidants

An antioxidant functions, among other things, to scavenge free radicals from skin to protect the skin from environmental aggressors. Examples of antioxidants that may be used in the present compositions include, but are not limited to, compounds having phenolic hydroxy functions, such as ascorbic acid and its derivatives/esters; beta-carotene; catechins; curcumin; ferulic acid derivatives (e.g. ethyl ferulate, sodium ferulate); gallic acid derivatives (e.g. propyl gallate); lycopene; reductic acid; rosmarinic acid; tannic acid; tetrahydrocurcumin; tocopherol and its derivatives; uric acid; and mixtures thereof. Other suitable antioxidants are those that have one or more thiol functions (—SH), in both reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur.

### Deodorizing Agents

Deodorizing agents counteract, mask or eliminate body odors that are formed through the action of skin bacteria on apocrine perspiration which results in the formation of unpleasant smelling degradation products. Accordingly, suitable deodorizing agents include, inter alia, microbial inhibitors, enzyme inhibitors, odor absorbers and odor maskers. Undecylenic acid and ester derivatives of undecylenic acid have been found to possess significant deodorizing activity. Polyoxyalkylene and simple alkyl esters of undecylenic acid (e.g., methyl undecylenate and ethyl undecylenate) are well-known deodorizers. Esterase inhibitors such as the trialkyl citrates (e.g., trimethyl citrates, triethyl citrates, tripropyl citrates, triisopropyl citrates, tributyl citrates) are useful deodorizers. Additional examples of useful esterase inhibitors are sterol sulfates and phosphates, such as, for example, lanosterine-, cholesterine-, campesterine-, stigmasterine- and sitosterine sulfates and phosphates, respectively; dicarbonic acids and their esters, such as, for example, glutaric acid, glutaric acid monoethylester, glutaric acid diethylester, adipinic acid, adipinic acid monoethylester, adipinic acid diethylester, malonic acid and malonic acid diethylester, hydroxycarbonic acids and their esters such as, for example, citric acid, malonic acid, tartaric acid or tartaric acid diethylester. Other deodorizing agents include deodorant compounds (e.g., 2-amino-2-methyl-1-propanol (AMP), ammonium phenolsulfonate; benzalkonium chloride; benzethonium chloride, bromochlorophene, cetyltrimethylammonium bromide, cetyl pyridinium chloride, chlorophyllin-copper complex, chlorothymol, chloroxylenol, cloflucarban, dequalinium chloride, dichlorophene, dichloro-mxylenol, disodium dihydroxyethyl sulfosuccinylundecylenate, domiphen bromide, hexachlorophene, lauryl pyridinium chloride, methylbenzethonium chloride, phenol, sodium bicarbonate, sodium phenolsulfonate, triclocarban, triclosan, zinc phenolsulfonate, zinc ricinoleate, and mixtures thereof); and suitable mixtures of any of the above.

### pH Adjusting Agents

The pH of the compositions of the present technology can be adjusted with any combination of acidic and/or basic pH adjusting agents known to the art. Acidic materials include organic acids and inorganic acids, for example, acetic acid, citric acid, tartaric acid, alpha-hydroxy acids, beta-hydroxy acids, salicylic acid, lactic acid, glycolic acid, and natural fruit acids, or inorganic acids, for example, hydrochloric acid, nitric acid, sulfuric acid, sulfamic acid, phosphoric acid, and combinations thereof.

Basic materials include inorganic and organic bases, and combinations thereof. Examples of inorganic bases include but are not limited to the alkali metal hydroxides (especially sodium, potassium, and ammonium), and alkali metal salts such as sodium borate (borax), sodium phosphate, sodium pyrophosphate, and the like; and mixtures thereof. Examples of organic bases include but are not limited to triethanolamine (TEA), diisopropanolamine, triisopropanolamine, aminomethyl propanol, dodecylamine, cocamine, oleamine, morpholine, triamylamine, triethylamine, tetrakis(hydroxypropyl)ethylenediamine, L-arginine, aminomethyl propanol, tromethamine(2-amino 2-hydroxymethyl-1,3-propanediol), and PEG-15 cocamine.

### Buffer Agents

Buffering agents can be used in the compositions of the technology. Suitable buffering agents include, but are not limited to, alkali or alkali earth metal carbonates, phosphates, bicarbonates, citrates, borates, acetates, acid anhydrides, succinates, and the like, such as sodium phosphate, sodium citrate, sodium acetate, sodium bicarbonate, and sodium carbonate.

The pH adjusting agent(s) and/or buffering agent is utilized in any amount necessary to obtain and/or maintain a desired pH value in the composition. The pH of the emulsions of the technology range from about 2 to about 10 in one aspect, from about 3 to about 9 in another aspect, and from about 3.5 to about 8 in a further aspect.

### Chelation Agents

Chelating agents can be employed to stabilize the personal care, home care, health care, and institutional care compositions of the technology against the deleterious effects of metal ions. When utilized, suitable chelating agents include EDTA (ethylene diamine tetraacetic acid) and salts thereof such as disodium EDTA, citric acid and salts thereof, cyclodextrins, and the like, and mixtures thereof.

### Structuring Agents

The compositions of the present technology may contain a structuring agent. Structuring agents are especially suitable in the emulsions of the present technology, for example, in the oil-in-water emulsions of the present technology. Without being limited by theory, it is believed that the structuring agent assists in providing rheological characteristics (for example yield and structural characteristics) to the composition which contribute to the stability of the composition.

The structuring agents of the present technology may be selected from stearic acid, palmitic acid, stearyl alcohol, cetyl alcohol, behenyl alcohol, palmitic acid, the polyethylene glycol ether of stearyl alcohol having an average of from about 1 to about 5 ethylene oxide units, the polyethylene glycol ether of cetyl alcohol having an average of from about 1 to about 5 ethylene oxide units, and mixtures thereof. In one aspect, structuring agents of the present technology are selected from the group consisting of stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof. In another aspect, structuring agents are selected from the group consisting of stearic acid, palmitic acid, stearyl alcohol, cetyl alcohol, behenyl alcohol, steareth-2, and mixtures thereof.

### Deposition Aids

The deposition aids of the compositions of the present technology can be selected from polymers which carry a cationic charge. The polymers are cationic derivatives of natural and synthetic polymers. In one aspect the naturally derived cationic polymers are derivatives of polygalactomannans such as guar and cassia gum. Suitable cationic guar gum derivatives are those having the INCI designation Guar Hydroxypropyltrimonium Chloride, available commercially from Rhodia Novecare under the trade names Jaguar® C13S, Jaguar® C15, Jaguar® C16 and Jaguar® C17 guar derivatives. Suitable cationic cassia derivatives having the INCI Name Cassia Hydroxypropyltrimonium Chloride are available from Lubrizol Advanced Materials, Inc. under the trade names Sensomer™ CT-250 and Sensomer™ CT-400 polymers. Other suitable deposition aids include quaternary nitrogen substituted cellulose ether derivatives INCI designation Polyquaternium-10 such as those commercially available under the Ucare™ JR-400, JR-125, JR-30M, LR-400, LR-30M and LK polymer series trade names from Dow Chemical Company.

Suitable cationic synthetically derived polymers for use in the compositions of the present technology contain cationic nitrogen-containing moieties such as quaternary ammonium or cationic protonated amino moieties. The cationic protonated amines can be primary, secondary, or tertiary amines (preferably secondary or tertiary), depending upon the particular species and the desired pH of the composition. Any anionic counterions can be used in association with the cationic polymers so long as the polymers remain soluble in the water phase of the composition and so long as the counterions are physically and chemically compatible with the essential components of the emulsified composition or do not otherwise unduly impair product performance, stability or aesthetics. Non-limiting examples of such counter-ions include halides (e.g., chlorine, fluorine, bromine, and iodine), sulfate and methylsulfate.

The cationic nitrogen containing moiety of the synthetic cationic polymer is generally present as a substituent on all, or more typically on some, of the monomer units thereof. Thus, the cationic polymer for use in the shampoo composition includes homopolymers, copolymers, terpolymers, and so forth, of quaternary ammonium or cationic amine substituted monomer units, optionally in combination with non-cationic monomers. Non-limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone. The alkyl and dialkyl substituted monomers have from C₁ to C₇ alkyl groups in one aspect and from C₁ to C₃ alkyl groups in another aspect. Other suitable monomers include vinyl esters, vinyl alcohol (made by hydrolysis of polyvinyl acetate), maleic anhydride, propylene glycol, and ethylene glycol.

Suitable cationic protonated amino and quaternary ammonium monomers, for inclusion in the cationic polymers of the shampoo composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C₁, C₂ or C₃ alkyls.

Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are C₁-C₇ hydrocarbyls in one aspect and C₁-C₃, alkyls in another aspect.

Other suitable cationic polymers for use in the emulsified compositions include copolymers of 1-vinyl-2-pyrrolidone and 1-vinyl-3-methylimidazolium salt (e.g., chloride salt) (INCI name: Polyquaternium-16) such as those commercially available from BASF Corporation under the LUVIQUAT™ trade name (e.g., product designations FC 370 and FC 905); copolymers of 1-vinyl-2-pyrrolidone and dimethylaminoethyl methacrylate (INCI name: Polyquaternium-11) such as those commercially available from Ashland Inc. under the GAFQUAT trade name (e.g., product designation 755N); cationic diallyl quaternary ammonium containing polymers, including, for example, dimethyldiallylammonium chloride homopolymer, copolymers of acrylamide and dimethyldiallylammonium chloride (INCI name: Polyquaternium 6 and Polyquaternium 7), such as those available under the MERQUAT™ trade name (e.g., product designations 100 and 550) from Lubrizol Advanced Materials, Inc.; amphoteric copolymers of acrylic acid including copolymers of acrylic acid and dimethyldiallylammonium chloride (INCI name: Polyquaternium-22) such as available from Lubrizol Advanced Materials, Inc. under the Merquat trade name (e.g., product designations 280 and 295), terpolymers of acrylic acid with dimethyldiallylammonium chloride and acrylamide (INCI name: Polyquaternium-39) such as those available from Lubrizol Advanced Materials, Inc. under the Merquat™ trade name (e.g., product designations 3300 and 3331), and terpolymers of acrylic acid with methacrylamidopropyl trimethylammonium chloride and methylacrylate (INCI name: Polyquaternium-47) available from Lubrizol Advanced Materials, Inc. under the Merquat™ trade name (e.g., product designation 2001).

The forgoing deposition aids serve a dual function in that they provide conditioning and sensory aesthetics to the hair and/or skin.

### UV Protection Agents

UV protective agents (UV-B and UV-A) are organic compounds which are able to absorb ultraviolet rays and release the absorbed energy in the form of longer wave radiation such as heat. The UV protective agents suitable for use herein may be classified into groups based upon their chemical structure: organic camphor derivatives, para-amino benzoates; salicylates; cinnamates; benzophenones; benzalmalonates, triazine derivatives, and miscellaneous compounds. The UV protective agents may be oil-soluble or water-soluble. Examples of oil soluble UV-B protective agents include, but are not limited to, 3-benzylidenecamphor and derivatives thereof, e.g., 3-(4-methylbenzylidene)camphor, 4-aminobenzoic acid derivatives, such as 2-ethylhexyl 4-(dimethylamino)benzoate, 2-ethylhexyl 4-(dimethylamino)benzoate and amyl 4-(dimethylamino)benzoate; esters of cinnamic acid, such as 2-ethylhexyl 4-methoxycinnamate, isopentyl 4-methoxycinnamate, 2-ethylhexyl 2-cyano-3-phenylcinnamate (octocrylene); esters of salicylic acid, such as 2-ethylhexyl salicylate, 4-isopropylbenzyl salicylate, homomenthyl salicylate; derivatives of benzophenone, such as 2-hydroxy-4-methoxybenzophenone, 2-hydroxy-4-methoxy-4′-methylbenzophenone, 2,2′-dihydroxy-4-methoxybenzophenone; esters of benzalmalonic acid, such as di-2-ethylhexyl 4-methoxybenzalmalonate; triazine derivatives, such as, for example, 2,4,6-trianilino (p-carbo-2′-ethyl-1′-hexyloxy)-1,3,5-triazine and octyltriazone; and propane-1,3-diones, such as 1-(4-tert-butylphenyl)-3-(4′-methoxyphenyl)propane-1,3-dione, and menthyl anthralinate and digalloyl trioleate.

Suitable water soluble UV-B protective agents include, but are not limited to, 2-phenylbenzimidazole-5-sulfonic acid and the alkali metal, alkaline earth metal, ammonium, alkylammonium, alkanolammonium and glucammonium salts thereof; sulfonic acid derivatives of benzophenone, such as 2-hydroxy-4-methoxybenzophenone-5-sulfonic acid and its salts; sulfonic acid derivatives of 3-benzylidenecamphor, such as 4-(2-oxo-3-bornylidenemethyl)benzenesulfonic acid, and 2-methyl-5-(2-oxo-3-bornylidene)sulfonic acid and salts thereof. Mixtures of the oil soluble and water soluble UV protective agents can be used in the emulsions of the technology.

Suitable typical UV-A protective agents include, but are not limited to, derivatives of benzoylmethane, such as1-(4′-tert-butylphenyl)-3-(4′-methoxyphenyl)propane-1,3-dione and 1-phenyl-3-(4′-isopropylphenyl)propane-1,3-dione. The UV-A and UV-B filters can of course also be used in mixtures. Additional UV protective agents are disclosed in U.S. Pat. Nos. 5,169,624; 5,543,136; 5,849,273; 5,904,917; 6,224,852; 6,217,852; and Segarin et al., chapter VII, pages 189 of Cosmetics Science and Technology, and Final Over-the-Counter Drug Products Monograph on Sunscreens (Federal Register, 1999:64:27666-27963), all of which are incorporated herein by reference.

### Sunscreens

The sunscreens are insoluble or particulate substances that provide a physical barrier against UV radiation on the skin. Insoluble pigments are suitable for this purpose, namely finely disperse metal oxides or salts, such as, for example, titanium dioxide, zinc oxide, iron oxide, aluminum oxide, cerium oxide, zirconium oxide, silicates (talc), barium sulfate and zinc stearate. In one aspect, the particles have an average diameter of less than 100 nm, between 5 and 50 nm in another aspect, and between 15 and 30 nm in still another aspect. Typically, they have a spherical shape, although it is also possible to use particles which have an ellipsoidal or plate-like shape or a shape which deviates in some other way from the spherical configuration. A relatively new class of photoprotective filters are micronized organic pigments, such as, for example, 2,2′-methylenebis{6-(2H-benzotriazol-2-yl)-4-(1,1,3,3-tetramethylbutyl)phenol} with a particle size of less than 200 nm.

### Insect Repellents

An insect repellent is any compound or composition which deters insects from a host. Suitable insect repellents useful in the compositions of the technology include, but are not limited to, N,N-diethyl-m-toluamide (DEET), pentane-1,2-diol or 3-(N-n-butyl-N-acetylamino)-propionic acid ethyl ester), dihydronepetalactone (DHN), butylacetylaminopropionate, natural pyrethroids such as the extract of the ground flowers of Chrysanthemum cinerariaefolium or C. coccineum, and citronella oil.

### Antiperspirants

Various antiperspirant agents that can be utilized according to the present technology include conventional antiperspirant metal salts and complexes of metal salts. In one aspect of the technology the metal salts and metal salt complexes utilized as the antiperspirant agents are acidic and are based on aluminum and zirconium and combinations thereof. These salts include but are not limited to aluminum halides, aluminum hydroxyhalides, aluminum sulfate, zirconium (zirconyl) oxyhalides, zirconium (zirconyl) hydroxyhalides, and mixtures or complexes thereof. Complexes of aluminum and zirconium salts include aluminum and zirconium salt complexes with amino acids, such as, for example, glycine or complexes with a glycol, such as, for example, propylene glycol (PG) or polyethylene glycol (PEG). Exemplary antiperspirant agents include but are not limited to aluminum chloride, aluminum chlorohydrate, aluminum dichlorohydrate, aluminum sesquichlorohydrate, zirconyl hydroxychloride, aluminum chlorohydrex PEG (aluminum chlorohydrex polyethylene glycol), aluminum chlorohydrex PG (aluminum chlorohydrex propylene glycol), aluminum dichlorohydrex PEG (aluminum dichlorohydrex polyethylene glycol), aluminum dichlorohydrex PG (aluminum dichlorohydrex propylene glycol), aluminum sesquichlorohydrex PEG (aluminum sesquichlorohydrex polyethylene glycol), aluminum sesquichlorohydrex PG (aluminum sesquichlorohydrex propylene glycol), aluminum zirconium trichlorohyrate, aluminum zirconium tetrachlorohyrate, aluminum zirconium pentachlorohyrate, aluminum zirconium octachlorohyrate, aluminum zirconium chlorohydrex GLY (aluminum zirconium chlorohydrex glycine), aluminum zirconium trichlorohydrex GLY (aluminum zirconium trichlorohydrex glycine), aluminum zirconium tetrachlorohyrex GLY (aluminum zirconium tetrachlorohyrex glycine), aluminum zirconium pentachlorohyrex GLY (aluminum zirconium pentachlorohyrex glycine), and aluminum zirconium octachlorohyrex GLY (aluminum zirconium octachlorohyrex glycine). Other antiperspirant agents include ferric chloride and zirconium powder. Mixtures of any of the foregoing antiperspirant agents are also suitable for use in the present technology.

### Pharmaceutical and Cosmeceutical Actives

The compositions of the present technology can be formulated with at least one skin and/or hair care active (e.g., pharmaceutical and/or a cosmeceutical active compound to deliver a desired benefit effect when topically applied to the skin, hair and nails). These compounds may be soluble in oil or water, and may be present primarily in the oil phase or the water phase of the present compositions. Suitable benefit agents include, but are not limited to, vitamins, peptides, sugar amines, oil control agents, self-tanning actives, anti-acne actives, desquamation actives, skin lightening agents, depilatories, astringents, flavonoids, protease inhibitors, hair growth stimulating agents, anticellulite agents, anti-stretch mark actives, anti-wrinkle actives, lip plumping agents, anti-inflammatory and analgesic agents, anti-microbial and anti-fungal actives, and combinations thereof.

As used herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C₁-C₁₈ nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition may comprise a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

The composition may comprise one or more peptides. As used herein, “peptide” refers to peptides containing ten or fewer amino acids, their derivatives, isomers, and complexes with other species such as metal ions (for example, copper, zinc, manganese, and magnesium). Peptide refers to both naturally occurring and synthesized peptides. In one aspect, the peptides are di-, tri-, tetra-, penta-, and hexa-peptides, their salts, isomers, derivatives, and mixtures thereof. Peptide derivatives also useful herein include lipophilic derivatives, e.g., palmitoyl derivatives, such as, for example, palmitoyl-lys-thrthr-lys-ser, palmitoyl-gly-his-lys, their derivatives, and combinations thereof. Examples of useful peptide derivatives include, but are not limited to, peptides derived from soy proteins, carnosine (beta-alanine-histidine), palmitoyl-lysinethreonine (pal-KT) and palmitoyl-lysine-threonine-threonine-lysine-serine (palKTTKS, available in a composition known as MATRIXYL®), palmitoyl-glycineglutamine-proline-arginine (pal-GQPR, available in a composition known as RIGIN®), these three being available from Sederma, France, acetyl-glutamateglutamate-methionine-glutamine-arginine-arginine (Ac-EEMQRR; Argireline), and Cu-histidine-glycine-glycine (Cu-HGG, also known as IAMIN®). The compositions may comprise from about 1×10⁻⁷% to about 20%, alternatively from about 1×10⁻⁶% to about 10%, and alternatively from about 1×10⁻⁵% to about 5% of the peptide.

The composition may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Examples of sugar amines include glucosamine, N-acetyl glucosamine, mannosamine, N-acetyl mannosamine, galactosamine, N-acetyl galactosamine, their isomers (e.g., stereoisomers), and their salts (e.g., HCl salt).

The composition may comprise one or more compounds for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof.

The composition may comprise one or self-tanning actives for giving the skin an artificial suntanned appearance. Examples of self-tanning compounds are mono- or polycarbonyl compounds such asisatin, alloxan, ninhydrin, glyceraldehyde, mesotartaric aldehyde, glutaraldehyde, erythrulose, tyrosine, tyrosine esters, and dihydroxyacetone (DHA), 1,3,-dihydroxy-2-propanone).

Exemplary anti-acne compounds include acidic agents such as alpha-hydroxy acids (AHAs), beta-hydroxy acids (BHAs), alpha amino acids, alpha-keto acids (AKAs), acetic acid, azelaic acid, and mixtures thereof. Other anti-acne compounds include resorcinol, sulfur, salicylic acid, erythromycin, zinc, and benzoyl peroxide. Suitable anti-acne actives are described in further detail in U.S. Pat. No. 5,607,980.

The composition may comprise a safe and effective amount of a desquamation active for improving the texture and smoothness of the skin. Suitable examples comprise sulfhydryl compounds and zwitterionic surfactants which are described in U.S. Pat. No. 5,681,852.

The composition may comprise a skin-lightening agent. Suitable skin-lightening agents include kojic acid, arbutin, tranexamic acid, ascorbic acid and derivatives (e.g., magnesium ascorbyl phosphate or sodium ascorbyl phosphate or other salts of ascorbyl phosphate), ascorbyl glucoside, fatty esters of ascorbyl acid, such as ascorbyl palmitate, ascorbyl stearate, and the like Other suitable skin lightening materials include undecylenoyl phenylalanine aloesin, kojic acid, hydroquinone, arbutin, fruital, vegetal or plant extracts, such as lemon peel extract, chamomile, green tea, paper mulberry extract, and the like.

The composition may comprise a depilatory such as calcium and sodium hydroxide, calcium or sodium thioglycolate, and mixtures thereof.

The composition may comprise an astringent such as alum, oatmeal, yarrow, witch hazel, bayberry, and isopropyl alcohol.

The composition compositions may comprise a flavonoid. The flavonoid can be a synthetic material or obtained as extracts from natural sources, which also further may be derivatized. Examples of flavonoids suitable for use in the present technology are flavanones selected from unsubstituted flavanones, monosubstituted flavanones, and mixtures thereof; chalcones selected from unsubstituted chalcones, monosubstituted chalcones, disubstituted chalcones, trisubstituted chalcones, and mixtures thereof; flavones selected from unsubstituted flavones, monosubstituted flavones, disubstituted flavones, and mixtures thereof; one or more isoflavones; coumarins selected from unsubstituted coumarins, monosubstituted coumarins, disubstituted coumarins, and mixtures thereof; chromones selected from unsubstituted chromones, monosubstituted chromones, disubstituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with hydroxyl, C₁-C₈ alkyl, C₁-C₄ alkoxyl, O-glycoside, and the like or a mixture of these substituents.

Specific examples of suitable flavonoids include, but are not limited to, unsubstituted flavanone, mono-hydroxy flavanones (e.g., 2′-hydroxy flavanone, 6-hydroxy flavanone, 7-hydroxy flavanone, etc.), mono-alkoxy flavanones (e.g., 5-methoxy flavanone, 6-methoxy flavanone, 7-methoxy flavanone, 4′-methoxy flavanone, etc.), unsubstituted chalcone (especially unsubstituted trans-chalcone), mono-hydroxy chalcones (e.g., 2′-hydroxy chalcone, 4′-hydroxy chalcone, etc.), di-hydroxy chalcones (e.g., 2′,4-dihydroxy chalcone, 2′,4′-dihydroxy chalcone, 2,2′-dihydroxy chalcone, 2′,3-dihydroxy chalcone, 2′,5′-dihydroxy chalcone, etc.), and tri-hydroxy chalcones (e.g., 2′,3′,4′-trihydroxy chalcone, 4,2′,4′-trihydroxy chalcone, 2,2′,4′-trihydroxy chalcone, etc.), unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), unsubstituted coumarin, 4-hydroxy coumarin7-hydroxy coumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, and mixtures thereof. Additional examples of suitable flavonoids are also disclosed in U.S. Pat. No. 6,235,773.

The composition may comprise protease inhibitors including, but are not limited to, hexamidine compounds (e.g., hexamidine diisethionate), vanillin acetate, menthyl anthranilate, soybean trypsin inhibitor, Bowman-Birk inhibitor, and mixtures thereof.

Hair growth stimulating agents may comprise any agent which stimulates hair growth and/or prevents hair loss, or thinning, including but are not limited to polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, siNA, siRNA, dsRNA, dsDNA, anti-senseDNA, nucleic acids, synthetic molecules, and combinations thereof, which may be employed to achieve the therapeutic effect on stimulation of hair growth and/or prevention of loss of hair e.g., the eyelid, eyebrow, scalp, and skin.

Non-limiting examples of prostaglandins are those of the A, F and E types. Prostaglandin derivatives that exhibit high pharmacological activity and no or only very small side effects, such as 13,14-dihydro-15-dehydro-17-phenyl-18,19,20-trinor-PGF2α and its carboxylic acid esters.

Examples of pharmaceutical hair growth stimulating agent and/or hair growth stimulating agent and/or hair density increasing agent and/or hair loss prevention agents include, but are not limited to prostaglandin A2, prostaglandin F2α, prostacyclin, prostaglandin E1, prostaglandin E2, 7-thiaprostaglandin E1, 16,17,18,19,20-pentanol-15-cyclohexyl-7-thiaprostaglandin E1, 16,17,18,19,20-pentanol-15-cyclopentyl-7-thiaprostaglandin E1, 16,16-dimethyl-7-thiaprostaglandin E1, 17,20-dimethyl-7-thiaprostaglandin E1, 16,17,18,19,20-pentanol-15-cyclohexyl-δ2-7-thiaprostaglandin E1, 16,16-dimethyl-52-prostaglandin E1, 7-fluoroprostacyclin, 5-fluoroprostacyclin, 16,17,18,19,20-pentanol-15-cycrohexylprostacyclinor 16,17,18,19,20-pentanol-15-cycropentylprostacyclin. Other examples of prostaglandins and prostaglandin analogues which may be used in the instant technology include, but are not limited to, Arbaprostil, Carboprost, Enprostil, Bimatoprost, Bemeprost, Latanaoprost, Limaprost, Misoprostol, Minoxidil, Ornoprostil, Prostacyclin, Prostaglandin E1, Prostaglandin E2, Prostaglandin F2α, Rioprostil, Rosaprostol, Sulprostone, Travaprost, Trimoprostil, and Viprostol. Other examples of hair growth stimulating and/or hair loss prevention agents include, but are not limited to 15-hydroxyprostaglandin dehydrogenase (15-PGDH) inhibitors, including but not limited to pyrazolecarboxamide compounds, tetrazole compounds, and 2-alkylideneaminooxyacetamide compounds (see U.S. Patent Application Publication Numbers 2006/0026775, 2004/0052760, and 2004/0235831).

Other non-limiting examples of hair growth stimulating agents are hexamidine, butylated hydroxytoluene (BHT), hexanediol, panthenol and pantothenic acid derivatives, their isomers, salts and derivatives, and mixtures thereof.

The composition may comprise anti-stretch mark actives including arabignogalactan, lupeol, soya peptides, tripeptides composed of the amino acids glycine, histidine and lysine, sophora (Sophora japonica) flower extract, chlorophyceae (Enteromorpha compressa) extract, peptide extract of avocado, panthenol, and mixtures thereof.

The composition may comprise an effective amount of an anticellulite agent. Suitable agents may include, but are not limited to, xanthine compounds caffeine, theophylline, theobromine, and aminophylline.

The compositions of the present technology may further contain one or more anti-wrinkle actives. Exemplary anti-wrinkle actives suitable for use include retinol and retinol derivatives, sulfur-containing D- and L-amino acids and their derivatives and salts, particularly the N-acetyl derivatives, a preferred example of which is N-acetyl-DL-Methionine; thiols, e.g., ethane thiol; hydroxy acids, e.g., alpha-hydroxy acids such as lactic acid and glycolic acid or beta-hydroxy acids such as salicylic acid and salicylic acid derivatives such as the dodecylamide; stilbenes; hydroxystilbenes; hyaluronic acid; flavonoids; xanthones; Beta-Glucans; Skleroglucan; triterpenoid acids, e.g., arjunolic acid, ursolic acid); turmeric oil; xymeninic acid; creatine; sphingolipids such as salicycloyl-phytosphingosine, phytosphingosine, sphingosine, sphinganine and their derivatives; phytic acid; lipoic acid; lysophosphatidic acid; skin peel agents, e.g., phenol and the like); vitamin B3 compounds and retinoids.

The composition may comprise any agent that has a temporary or permanent lip plumping effect, for instance menthol, capsaicinoids, vanillyl butyl ether, capsicum, niacin, menthol, caffeine, or extracts of peppermint, ginger, clove, cinammon, or ginseng, or a peptide based material such as for instance hexapeptide-3. Temporary lip plumping agents may optionally work by causing irritation to the lip tissue, whereas longer lasting or more permanent effects may be observed from agents that modify the collagen or moisture composition of lips.

The composition may comprise anti-inflammatory and analgesic actives. The anti-inflammatory agent can be utilized for its aesthetic and/or therapeutic benefit when topically applied to the skin. In one aspect, anti-inflammatory agents can enhance the skin appearance benefits of the present technology, e.g., such agents, contribute to a more uniform and acceptable skin tone or color. In another aspect, anti-inflammatory/analgesic agents are utilized therapeutically to reduce pain and/or swelling. Anti-inflammatory/analgesic agents can be classified into steroidal and non-steroidal agents. Specific steroidal and non-steroidal anti-inflammatory/analgesic agents useful in the composition technology include, but are not limited to, bisabolol, allantoin, phytantriol, coenzyme Q10, licorice extract, nicotinate esters, capsaicin and capsicum extracts and derivatives, glycyrrhizidine and idebenone, aspirin, ibuprofen, ketoprofen, piroxicam, flurbiprofen, naproxen, diclofenac, felbinac, and combinations thereof. The exact amount of anti-inflammatory agent to be used in the compositions will depend on the particular anti-inflammatory agent utilized since such agents vary widely in potency.

The compositions of the present technology may contain an antimicrobial or antifungal active. Such actives are capable of destroying microbes, preventing the development of microbes or preventing the pathogenic action of microbes. Examples of antimicrobial and antifungal actives include beta-lactam drugs, guanidinium compounds, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, N-octyl lactamide, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione and clotrimazole, and combinations thereof.

### Skin and Hair Conditioners

The compositions of the present technology may comprise from about 0.1% to about 50%, alternatively from about 0.5% to about 30%, alternatively from about 1% to about 20%, alternatively from about 2% to 15%, of a conditioning agent. These conditioning agents include, but are not limited to, hydrocarbon oils and waxes, silicones (volatile and nonvolitile, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof.

Non-limiting examples of hydrocarbon oils and waxes suitable for use as a conditioner include petrolatum, mineral oil, microcrystalline waxes, polyalkenes, paraffins, cerasin, ozokerite, polyethylene, perhydrosqualene, polyalphaolefins, hydrogenated polyisobutenes and combinations thereof.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference. Silicone fluids suitable for use as conditioners are disclosed in U.S. Pat. Nos. 2,826,551; 3,964,500; 4,364,837; 5,104,646; 5,106,609; U.S. Reissue Pat. No. 34,584; and British Patent No. 849,433, all of which are incorporated herein by reference.

### Preservatives

In one aspect, any preservative suitable for use in personal care, home care, health care, and institutional and industrial care products, can be used in the compositions of the present technology. Suitable preservatives include polymethoxy bicyclic oxazolidine, methyl paraben, ethyl paraben, propyl paraben, butyl paraben, benzyltriazole, DMDM hydantoin (also known as 1,3-dimethyl-5,5-dimethyl hydantoin), imidazolidinyl urea, phenoxyethanol, phenoxyethylparaben, methylisothiazolinone, methylchloroisothiazolinone, benzoisothiazolinone, triclosan, and suitable polyquaternium compounds disclosed above (e.g., Polyquaternium-1).

In another aspect, acid based preservatives are useful in the compositions of the present technology. The use of acid based preservatives facilitates the formulation of products in the low pH range. Lowering the pH of a formulation inherently provides an inhospitable environment for microbial growth. Moreover, formulating at low pH enhances the efficacy of acid based preservatives, and affords a personal care product which maintains an acidic pH balance on the skin as discussed by Wiechers, 2008.

Any acid based preservative that is useful in personal care, home care, health care, and institutional and industrial care products can be used in the compositions of the present technology. In one aspect, the acid preservative is a carboxylic acid compound represented by the formula: R⁵³C(O)OH, wherein R⁵³ represents hydrogen, a saturated and unsaturated hydrocarbyl group containing 1 to 8 carbon atoms or C₆ to C₁₀ aryl. In another aspect, R⁵³ is selected from a hydrogen, a C₁ to C₈ alkyl group, a C₂ to C₈ alkenyl group, or phenyl. Exemplary acids are, but are not limited to, formic acid, acetic acid, propionic acid, sorbic acid, caprylic acid, and benzoic acid, and mixtures thereof.

In another aspect, suitable acids include but are not limited to, oxalic acid, succinic acid, glutaric acid, adipic acid, azelaic acid, maleic acid, fumaric acid, lactic acid, glyceric acid, tartronic acid malic acid, tartaric acid, gluconic acid, citric acid, ascorbic acid, salicylic acid, phthalic acid, mandelic acid, benzilic acid, and mixtures thereof. The acid based preservatives and/or their salts can be used alone or in combination with non-acidic preservatives typically employed in personal care, home care, health care, and institutional and industrial care products.

Salts of the foregoing acids are also useful as long as they retain efficacy at low pH values. Suitable salts include the alkali metal (e.g., sodium, potassium, calcium) and ammonium salts of the acids enumerated above.

### Electrolytes

The emulsion composition may contain an electrolyte. Suitable electrolytes are known compounds and include salts of multivalent anions, such as potassium pyrophosphate, potassium tripolyphosphate, and sodium or potassium citrate, salts of multivalent cations, including alkaline earth metal salts such as calcium chloride and calcium bromide, as well as zinc halides, barium chloride and calcium nitrate, salts of monovalent cations with monovalent anions, including alkali metal or ammonium halides, such as potassium chloride, sodium chloride, potassium iodide, sodium bromide, and ammonium bromide, alkali metal or ammonium nitrates.

Oil in water emulsion based formulations prepared with the multi-functional additives as disclosed herein can be highly concentrated emulsions that are stable against flocculation and coalescence. The emulsions are completely stable on storage even at elevated temperature (up to about 50° C.). Emulsion based formulations prepared with the multi-functional additives as disclosed herein can remain colloidally stable dispersions even as highly concentrated hydrophobic medium O/W emulsions, where the volume fraction of the hydrophobic medium in the emulsion can be as high as 0.2 in one aspect, as high as 0.30 in another aspect, as high as 0.40 in still another aspect, as high as 0.50 in a further aspect, as high as 0.55 in a still further aspect, and as high as 0.60 in an additional aspect. By “volume fraction” it is meant the ratio of the volume of the hydrophobic medium to the total volume of the emulsion (hydrophobic and hydrophilic medium). In another exemplary embodiment, the volume fraction of the hydrophobic medium ranges from about 0.01 to about 0.60 in one aspect, from about 0.1 to about 0.50 in another aspect, from about 0.15 to about 0.40 in still another aspect, from about 0.25 to about 0.30 in a further aspect, and from about 0.35 to about 0.20 in an additional aspect based on the total volume of the emulsion.

In another embodiment, the multi-functional additive can comprise up to about 50 wt. % of the hydrophobic phase in one aspect, up to about 60 wt. % in another aspect and up to about 75 wt. % in still another aspect, up to about 85 wt. % in a further aspect, up to about 90 wt. % in a still further aspect, and up to about 95 wt. % in an additional aspect based on the total weight of the emulsion (oil and water). In another exemplary embodiment, the hydrophobic phase component ranges from about 1 to about 95 wt. % in one aspect, from about 5 to about 85 wt. % in another aspect, from about 10 to about 40 wt. % in still another aspect, and from about 15 to about 30 wt. % in a further aspect, based on the total weight of the oil, water and emulsifier components.

The multi-functional additives can be used in a wide variety of applications such as, for example, in personal care, cosmetics, home care, food, health care, and/or institutional and industrial care products.

The term “personal care” as used herein includes, without being limited thereto, cosmetics, toiletries, cosmeceuticals, beauty aids, sunscreens, insect repellents, personal hygiene and cleansing products applied to the body, including the skin, hair, scalp, and nails of humans and animals.

The term “home care products” as used herein includes, without being limited thereto, products employed in a domestic household for surface cleaning or maintaining sanitary conditions, such as in the kitchen and bathroom (e.g., hard surface cleaners, hand and automatic dish care, toilet bowl cleaners and disinfectants), and laundry products for fabric care and cleaning (e.g., detergents, fabric conditioners, pretreatment stain removers), and the like.

The term “health care” as used herein includes, without being limited thereto, pharmaceuticals (controlled release pharmaceuticals), pharmacosmetics, oral care (mouth and teeth) products, such as oral suspensions, mouthwashes, toothpastes, dentifrices, and the like, and over the counter products and appliances (topical and transdermal), such as patches, plasters and the like, externally applied to the body, including the skin, scalp, nails and mucous membranes of humans and animals, for ameliorating a health-related or medical condition, for generally maintaining hygiene or well-being, and the like.

The term “institutional and industrial care” (“I&I”) as used herein includes, without being limited thereto, products employed for surface cleaning or maintaining sanitary conditions in institutional and industrial environments, textile treatments (e.g., textile conditioners, carpet and upholstery cleaners), automobile care (e.g., hand and automatic car wash detergents, tire shines, leather conditioners, liquid car polishes, plastic polishes and conditioners), paints and coatings, and the like.

In one aspect of the technology, a multi-functional additive according as described above can be employed to produce colloidally stable dispersions employed in personal care or cosmetic compositions. These are compositions which, through the use of O/W emulsions, are given a readily spreadable consistency due to the readily incorporated hydrophobic medium into the hydrophilic medium, for example, in creams, such as, skin and hair care creams, baby creams or sun protection creams, ointments, lotions or make-ups; in health care preparations, such as, ointments or creams; or in home care formulations, such as in pastes, waxes, polishes, and the like.

Non-limiting examples of personal care and cosmetic compositions in which the multi-functional additive may be employed to prepare a colloidally stable dispersion may be skin moisturizing creams, lotions, and sprays, anti-aging creams, lotions, and sprays, sunscreen creams, lotions, and sprays, skin lightening creams, lotions, and sprays self-tanning creams, lotions, and sprays, anti-acne creams, lotions, and sprays, skin exfoliating creams, lotions, and sprays, color cosmetic creams, lotions, and sprays, including liquid make-up and foundation, hair conditioning creams, lotions, and sprays, hair styling creams, lotions, and sprays, antiperspirant and deodorant gels, creams, lotions, and sprays, depilatory and shaving creams, lotions, and sprays, and hair coloring creams, lotions, and sprays.

Compositions including the multi-functional additive, such as a personal care or cosmetic composition, can optionally comprise other solid particulate or liquid benefit agents, such as, for example, fragrances, perfumes, botanicals, particulate materials (e.g., exfoliants, and anti-dandruff agents), insoluble materials, opacifiers and pearlizing agents, humectants, emollients, antioxidants, deodorizing agents, pH adjusting agents, buffers, chelation agents, viscosity modifiers, structuring agents, deposition aids, and topically active compounds such as UV protection agents, sunscreens, insect repellents, antiperspirants, cosmeceuticals, pharmaceuticals, skin and hair conditioners, preservatives, and combinations thereof, as described more particularly above with respect to the multi-functional additive.

When utilized in a composition, each optional component(s) typically is included in an amount of from about 0.0001 to about 90 wt. % in one aspect, from about 0.01 to 70 wt. % in another aspect, from about 0.025 to 50 wt. %, from about 0.05 to about 30 wt. %, from about 0.1 to about 15 wt. % in a still another aspect, from about 0.5 to about 10 wt. % in a further aspect, and from about 1 to about 5 wt. % in a still further aspect, based on the total weight of the composition. The amounts employed will vary with the purpose and character of the composition and can be readily determined by one skilled in the formulation arts and from the literature.

It is known that some of the materials described above may interact in the final formulation, so that the components of the final formulation may be different from those that are initially added. The products formed thereby, including the products formed upon employing the composition of the present technology in its intended use, may not be susceptible of easy description. Nevertheless, all such modifications and reaction products are included within the scope of the present technology; the present technology encompasses the composition prepared by admixing the components described above.

### EXAMPLES

### Example 1

This example shows that the galactomannan polymer, glyceryl cassia gum, has a relatively low surface-activity. Surface-activity is a measure of a compound's ability to adsorb at an oil-water interface, and hence a key requirement for a compound to function as an emulsifier.

Herein, surface-activity is measured by measuring the surface tension of an aqueous solution of glyceryl cassia gum, using the Wilhelmy plate method for surface-tension measurement, allowing a time-period of 1 minute, called herein the equilibration time. Highly surface-active compounds, when dissolved in deionized water at an amount of about 1% (w/w) or less, can typically reduce the equilibrium surface tension value from about 72 dyne/cm, that of deionized water, to about 50 dyne/cm or lower. Table I shows the surface tension values for different concentrations of glyceryl cassia gum in aqueous solutions.

TABLE I

| | Surface Tension, |
|---|---|
| Sample | Dyne/cm |
| Deionized water | 72.27 |
| 0.1% (w/w) Glyceryl Cassia Gum in deionized | 71.81 |
| water | |
| 0.5% Glyceryl Cassia Gum in deionized water | 68.95 |
| 1% Glyceryl Cassia Gum in deionized water | 67.8 |

It may be noted that, even when surface-tension measurements were carried out with an increased equilibration time, the surface tension of a 1% solution of glyceryl cassia gum was 59.91 dyne/cm, measured after an equilibration time of 30 minutes. These results suggest that glyceryl cassia gum alone would not meet the object of the present invention insofar as having a multifunctional additive providing emulsifier-functionality for oil droplets in oil-in-water emulsions.

### Example 2

This example shows that the adsorption of glyceryl cassia gum polymer on oil droplets (i.e., oil-water interface) in O-W emulsions increases significantly, in the presence of an amphiphilic anchoring-agent having hydrogen-bonding groups in the polar, or “hydrophilic” portion of the compound. It further shows that, in the absence of the amphiphilic anchoring agent, the amount of glyceryl cassia gum adsorbing on oil droplets in O-W emulsions is relatively low, thus providing an additional confirmation of the low surface-activity of the said galactomannan polymer.

Oil-in-water emulsions (180-g batch) were prepared by adding a given weight of an oil-phase comprising a mixture of octocrylene (2-ethylhexyl 2-cyano-3,3-diphenyl-2-propenoate) and octyl salicylate (both are oily UV-filter-actives used in sunscreens), wherein the weight-ratio of octocrylene to octyl salicylate is 65.5:34.5, to an aqueous solution of glyceryl cassia gum. An oil-soluble (water-insoluble), amphiphilic anchoring-agent, sorbitan oleate, was added to the oil-phase. The hydrophilic portion of the amphiphillic anchoring agent contains 9 hydrogen-bonding groups, including both hydrogen bond donors and acceptors, as shown in Formula I.

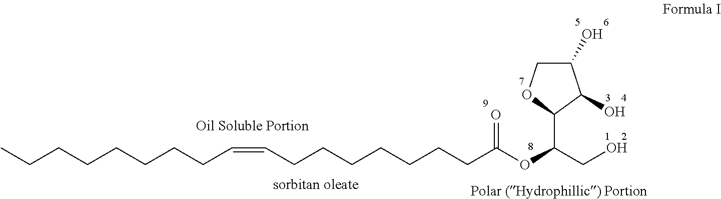

In producing the O-W emulsions, a Caframo mixer fitted with a dispersion-blade agitator was used for shearing the emulsions. Upon addition of the oil-phase to the aqueous-phase, shearing of the emulsions was continued for 20 minutes at a mixer-speed of 2,000 rpm. Thus produced, the amount of the oil-phase (anchoring-agent compound-free basis) in the foregoing emulsions was 16.7% (w/w).

In determining the amount of glyceryl cassia gum adsorbed on oil-droplets, the emulsions were heated to 60° C. for about 24 hours, following which they were centrifuged at 20,000 rpm, until a clear liquid phase (aqueous phase) separated from the rest of the emulsion at the top of centrifuge tubes. (The specific oil-phase used herein has a density that is greater than that of water, and accordingly the oil-phase or the emulsion-phase settles to the bottom of centrifuge tubes upon high-speed centrifuging.) The separated, clear aqueous-phase was pipetted out and analyzed for the amount of glyceryl cassia gum contained in it. Based on the total amount of glyceryl cassia gum added to the aqueous phase, and the amount left in the aqueous-phase after mixing the oil-phase with the aqueous-phase in producing the emulsion, the amount of glyceryl cassia gum adsorbed on oil droplets was calculated, based on a mass balance-calculation.

As shown in Table II, the adsorption of glyceryl cassia gum on oil droplets increases significantly in the presence of the amphiphilic anchoring agent.

TABLE II

| | Adsorbed Amount of |
|---|---|
| | Glyceryl Cassia Gum, |
| Emulsion Sample | mg/g of oil-phase |
| 0.83%* Glyceryl Cassia Gum + 0% | 5.86 |
| Sorbitan Oleate | |
| 0.83% Glyceryl Cassia Gum + 1.25% | 31.17 |
| Sorbitan Oleate | |
| 0.83% Glyceryl Cassia Gum + 2.5% | 32.22 |
| Sorbitan Oleate | |

*The total amount of glyceryl cassia gum in an emulsion, the rest of the emulsion being comprised of 16.7% by weight of the aforementioned oil-phase, a given amount of sorbitan oleate, as specified in Table II, and a balance-amount of deionized water.

### Example 3

This example shows that the coalescence-stability (stability against oil-separation) of oil droplets in oil-in-water (O-W) emulsions that contain a multifunctional additive of the present invention, comprising a mixture of glyceryl cassia gum and an amphiphilic anchoring agent compound with an AAEF value of greater than 1.8×10⁻³, is much greater than that of an emulsion that contains either glyceryl cassia gum or an amphiphilic anchoring-agent compound, but not any mixtures thereof. The higher stability of the emulsions is attributed to an increased adsorption of glyceryl cassia gum on the oil droplets in the presence of the amphiphilic anchoring agent. This example further shows that an amphiphilic anchoring agent, such as glycol stearate, whose AAEF value is less than 1.8×10⁻³, is not suitable for the multi-functional additive.

A series of O-W emulsions (180-g batch) were prepared using isocetyl stearate as the oil-phase, wherein a given amphiphilic anchoring agent was added to the oil-phase of an emulsion, if the compound is oil-soluble, or to the water-phase of an emulsion, if the compound is water-soluble. In producing these emulsions, both the oil-phase and the water-phase of the emulsions were heated to about 50-55° C., wherein the water-phase contained a given amount of glyceryl cassia gum dissolved in deionized water. The heated oil-phase was added to the heated water-phase under strong agitation, using a Caframo mixer fitted with a dispersion-blade agitator. The resulting emulsion was sheared at a mixer-speed of 1,000 rpm for about 3 minutes, followed by further shearing at a mixer-speed of 2,000 rpm for about 15 minutes.

Additional emulsions were prepared using a procedure similar to the one described above, wherein the emulsions did not contain glyceryl cassia gum in the water-phase, but contained an amphiphilic anchoring agent compound either in the oil-phase or in the water-phase of the emulsions.

Yet further emulsions were prepared, which did not contain any amphiphillic anchoring agent compound, but contained glyceryl cassia gum in the water-phase of the emulsion. The emulsion compositions are presented in Table III, along with the AAEF values of the various amphiphilic anchoring agents used therein.

TABLE III

| | | | | | Glyceryl | |
|---|---|---|---|---|---|---|
| | Amphiphilic | | Deionized | Isocetyl | Cassia | Anchoring |
| Emulsion | Anchoring | | Water, | stearate, | Gum, | Agent |
| Number | Agent | AEEF | Weight % | Weight % | Weight % | Weight % |
| E1 | Glyceryl | 0.002324 | 76.3 | 22.04 | 0.83 | 0.83 |
| | Stearate | | | | | |
| E2 | Sorbitan | 0.005199 | 76.3 | 22.04 | 0.83 | 0.83 |
| | Palmitate | | | | | |
| E3 | Sorbitan | 0.003134 | 76.3 | 22.04 | 0.83 | 0.83 |
| | Oleate | | | | | |
| E4 | Sorbitan | 0.003317 | 76.3 | 22.04 | 0.83 | 0.83 |
| | Stearate | | | | | |
| E5 | Methylglucose | 0.001835 | 76.3 | 22.04 | 0.83 | 0.83 |
| | Sesquistearate | | | | | |
| E6 | Sucrose | 0.009747 | 76.3 | 22.04 | 0.83 | 0.83 |
| | Cocoate | | | | | |
| E7 | Glycol | 0.001503 | 76.3 | 22.04 | 0.83 | 0.83 |
| | Stearate | | | | | |
| E8 | | | 76.95 | 22.22 | 0.83 | |
| E9 | Sorbitan | 0.003134 | 77.13 | 22.04 | | 0.83 |
| | Oleate | | | | | |
| E10 | Sorbitan | 0.003317 | 77.13 | 22.04 | | 0.83 |
| | Stearate | | | | | |
| E11 | Methylglucose | 0.001835 | 77.13 | 22.04 | | 0.83 |
| | Sesquistearate | | | | | |
| E12 | Sucrose | 0.009747 | 77.13 | 22.04 | | 0.83 |
| | Cocoate | | | | | |

The emulsions shown in Table III were tested for coalescence-stability by heating each emulsion to 60° C., and centrifuging the heated emulsion at 3,000 rpm for 30 minutes. All claimed emulsions (E1-E5) that contained a multifunctional additive (i.e., comprising a mixture of glyceryl cassia gum and an amphiphillic anchoring agent) showed reasonably high stability, with the centrifuged samples showing slight to no separation of the oil-phase-layer in the centrifuge tubes. Emulsion E6, which contained an amphiphillic anchoring-agent compound whose AAEF value is much less than 1.8×10⁻³, showed high separation (a thick layer in the centrifuge tubes) of the oil-phase. Emulsion E7, with no amphiphillic anchoring agent, also tested highly unstable showing a significantly high separation (thick layer) of the oil-phase. Similarly, emulsions E8-E11, which contained an amphiphillic anchoring-agent compound but not glyceryl cassia gum, showing a significantly high separation (thick layer) of the oil-phase.

### Example 4

This example shows the efficacious use of a dry-form of the multifunctional additive for producing an oil-in-water (O-W) emulsion that is highly stable against coalescence. The multi-functional additive contains a mixture of glyceryl cassia gum and methyl glucose sesquistearate, wherein the ratio of the weight of glyceryl cassia gum to the weight of methyl glucose sesquistearate is about 1:0.67 on a dry-basis (i.e., weight ratio of about 1.5 or greater). These components, as dry solids, may be added individually in producing O-W emulsions, or they may be blended into a homogeneous mixture, prior to addition to O-W emulsions.

Table IV shows the compositions of stable O-W emulsions comprising the aforementioned multi-functional additive, wherein the oil-phase comprises homomenthyl salicylate (homosalate), an oily UV-absorber-active used in sunscreen emulsions.

TABLE IV

| | | Emulsion Number | | |
|---|---|---|---|---|
| Phase | Ingredient | E1 | E2 | E3 |
| A | Glyceryl Cassia Gum, Weight % | 0.64 | 0.75 | 0.86 |
| A | Deionized Water, Weight % | 31.41 | 36.63 | |
| B | Methyl Glucose Sesquistearate | 0.43 | 0.50 | 0.57 |
| C | Deionized Water, Weight % | 46.14 | 40.74 | 35.28 |
| D | Oil-phase (Homosalate), Weight % | 21.38 | 21.38 | 21.38 |

The method for producing the foregoing emulsions (Batch size: 203.5 g) in Table IV is as follows.

i) Combine the Phase A ingredients in a suitable vessel, and mix until the glyceryl cassia gum solids dissolve completely
ii) Add the Phase B ingredient, heat the resulting mixture to 55° C. to melt the methyl glucose sesquistearate solids completely, and mix until homogeneous
iii) Cool the resulting mixture from (ii) to ambient temperature (about 20° C.)
iv) Add the Phase C ingredient, and mix until homogeneous
v) Transfer the mixture from (iv) to a rotor-stator homogenizer (Silverson), and while shearing the mixture at a homogenizer speed of 5,000 rpm, slowly add the Phase D ingredient, the oil-phase
vi) Continue shearing the resulting emulsion from (v) for about 10 minutes

### Example 5

This example demonstrates multi-functional additive formulations in which the weight-ratio of the weight of glyceryl cassia gum to the weight of the amphiphillic anchoring agent plays a role in the efficacy of the additive. Notably, the weight-ratio is expected to vary from one amphiphillic anchoring agent to another, and with a given amphiphillic anchoring agent it may vary depending on whether the multifunctional additive is added to a hydrophilic medium, i.e., the water-phase, or to the hydrophobic medium, i.e. the oil-phase, of an O-W emulsion.

A series of O-W emulsions were prepared according to the method described in EXAMPLE 4, varying the dosage of the multifunctional additive and hence of the glyceryl cassia gum, as well as the weight-ratio of glyceryl cassia gum to methyl glucose sesquistearate. The compositions of these emulsions are given in Table V, wherein the multifunctional additive was added to the water-phase of the emulsions.

TABLE V

| | Wt % | Weight-ratio | | | | |
|---|---|---|---|---|---|---|
| | Glyceryl | Glyceryl | | | | |
| | Cassia | Cassia to | Ingredient, Wt % | | | |
| | based on the | Methyl | | Methyl | | |
| Emulsion | Weight of | Glucose | Glyceryl | Glucose | Deionized | Oil- |
| Number | Oil-phase | Sesquistearate | Cassia | Sesquistearate | Water | phase |
| E1 | 3 | 1 | 0.64 | 0.64 | 77.34 | 21.38 |
| E2 | 3 | 1.25 | 0.65 | 0.52 | 77.45 | 21.38 |
| E3 | 3 | 1.5 | 0.64 | 0.43 | 77.55 | 21.38 |
| E4 | 3 | 2 | 0.64 | 0.32 | 77.66 | 21.38 |
| E5 | 3.5 | 1 | 0.75 | 0.75 | 77.12 | 21.38 |
| E6 | 3.5 | 1.25 | 0.75 | 0.60 | 77.27 | 21.38 |
| E7 | 3.5 | 1.5 | 0.75 | 0.50 | 77.37 | 21.38 |
| E8 | 3.5 | 2 | 0.75 | 0.37 | 77.50 | 21.38 |
| E9 | 4 | 1 | 0.85 | 0.85 | 76.92 | 21.38 |
| E10 | 4 | 1.25 | 0.86 | 0.69 | 77.07 | 21.38 |
| E11 | 4 | 1.5 | 0.86 | 0.57 | 77.19 | 21.38 |
| E12 | 4 | 2 | 0.86 | 0.43 | 77.33 | 21.38 |

The Brookfield viscosities of the foregoing O-W emulsions were measured, respectively, at 0.5, 1, 2.5, 5, 10, and 20 rpm of spindle speeds of a Brookfield RVT viscometer, and the ratio of 0.5 rpm-to-20 rpm viscosities, called herein the Shearing-Thinning Index, was calculated for each of the emulsions. Additionally, the viscosities of 1.1% (w/w), 0.96%, and 0.82% solutions of glyceryl cassia gum were measured at 0.5 and 20 rpm of spindle speeds. The concentrations of glycery cassia gum solutions correspond to the amounts of glyceryl cassia gum in Emulsions E1-E4, Emulsions E5-E8, and Emulsions E9-E12, respectively, based on the respective total average weights of glyceryl cassia gum and deionized water in these emulsions. For example, a weight of 100 g of Emulsions E1-E4 contains about 0.64 g of glyceryl cassia gum and about 77.5 g (averaged over the four emulsions) of deionized water; accordingly, the concentration of glyceryl cassia gum in these emulsions, based on the total average weights of glyceryl cassia gum and deionized water is 0.64/(0.64+77.5)*100=0.82%. The ratio of 0.5 rpm-to-20 rpm viscosities (i.e., the Shear-Thinning Index) was calculated for each of these glyceryl cassia gum solutions. Finally, the ratio of the Shear-Thinning Index of each of the emulsions to the Shear-Thinning Index of the corresponding glyceryl cassia gum solution (i.e., 0.82% solution of glyceryl cassia gum for Emulsions E1-E4, 0.96% solution for Emulsions E5-E8, and 1.1% solution for Emulsions E9-E12) were calculated, as presented in Table VI.

TABLE VI

| | Ratio of the Shear-thinning Index of an | | | |
|---|---|---|---|---|
| | Emulsion to the Shear-thinning Index of | | | |
| | the Corresponding Glyceryl Cassia Gum | | | |
| Glyceryl Cassia | Solution for Various Weight-ratios of | | | |
| Gum Dosage, | Glyceryl Cassia Gum and an Amphiphilic | | | |
| Weight %, | Anchoring-agent Compound | | | |
| based on the | Weight- | Weight- | Weight- | Weight- |
| oil-phase | ratio: 2 | ratio: 1.5 | ratio: 1.25 | ratio: 1 |
| 3 | 1.499 | 1.735 | 2.511 | 2.679 |
| | (Emulsion | (Emulsion | (Emulsion | (Emulsion |
| | E4) | E3) | E2) | E1) |
| 3.5 | 1.468 | 1.571 | 2.247 | 2.617 |
| | (Emulsion | (Emulsion | (Emulsion | (Emulsion |
| | E8) | E7) | E6) | E5) |
| 4 | 1.466 | 1.473 | 2.183 | 2.29 |
| | (Emulsion | (Emulsion | (Emulsion | (Emulsion |
| | E12) | E11) | E10) | E9) |

Generally, the Shear-Thinning Index of emulsions tends to increase significantly when emulsion droplets undergo high levels of flocculation. Increases in the Shear-thinning Index of O-W emulsions may also arise from an increase in the amount of the oil phase. For a given amount of an oil-phase in an emulsion, the Shear-Thinning Index may increase due to a reduction in the size of oil droplets, or from having a more uniform distribution of droplet-size, albeit, these effects on the Shear-Thinning Index are generally not as strong as that of flocculation of emulsion droplets. Accordingly, the results presented in Table VI suggest the possibility of high levels of flocculation in some of the O-W emulsions of Table V, wherein the weight-ratio of glyceryl cassia gum to amphiphilic anchoring agent, methyl glucose sesquistearate, is below 1.5 when the glyceryl cassia gum was first added in water.

### Example 6

This example demonstrates that a multifunctional additive comprising a mixture of glyceryl cassia gum and a pigment selected, for example, from zinc oxide, iron oxide, and titanium dioxide, can provide for pigment dispersancy functionality insofar as enabling deflocculation of pigment particles. Pigment dispersions were prepared adding a given amount of the multi-functional additive to water, and shearing the resulting dispersion at 2,750 rpm of mixer-speed for 10-12.5 minutes using a Caframo mixer fitted with a dispersion-blade agitator. The amounts of zinc oxide, iron oxide, and titanium dioxide in the respective dispersions (based on the total weight of pigment and water in the dispersions) were 20% (w/w), 22%, and 25%, respectively. The weight-ratio of glyceryl cassia gum to pigment in each of these dispersions was 0.02. Additional pigment dispersions (controls) were prepared, using a procedure similar to the one described above, with each of the three aforementioned pigments taken individually, wherein the individual dispersions contained the same weight of a given pigment, as noted above (e.g., 20% for zinc oxide), but did not contain glyceryl cassia gum.

The pigment dispersions comprising the multifunctional additive were diluted (10×) with a 0.056% (w/w) solution of glyceryl cassia gum, which had a thin (low-viscosity), water-like consistency. The control dispersions (i.e., free of glyceryl cassia gum) were diluted (10×) with deionized water. Dilution of the pigment dispersions was carried out by shearing a given pigment dispersion with a respective diluent, at 2,000 rpm of mixer-speed for 5 minutes, using a Caframo mixer fitted with a dispersion-blade agitator. The diluted dispersions were immediately centrifuged at 500 rpm for 30 minutes. Immediately afterwards, a dispersion sample was pipetted out from within the top 1-inch of the centrifuge tube. The extracted sample was then taken for a gravimetric analysis involving drying of the sample to a constant weight, measuring the weight of the dried sample, and calculating the weight % of solids in the extracted sample, the results of which are given in Table VII.

TABLE VII

| | Glyceryl Cassia | Weight % of |
|---|---|---|
| | Gum-to-Pigment | Solids in the |
| Pigment Dispersion | Weight-ratio | Extracted Sample |
| Iron Oxide (Control) | 0 | 0.0057 |
| Iron Oxide (Multifunctional | 0.02 | 1.2607 |
| Additive) | | |
| Titanium Dioxide (Control) | 0 | 0.2676 |
| Titanium Dioxide (Multifunctional | 0.02 | 1.0066 |
| Additive) | | |
| Zinc Oxide (Control) | 0 | 0.0099 |
| Zinc (Multifunctional Additive) | 0.02 | 1.3482 |

The much greater values of the weight % of solids for the extracted samples from the pigment dispersions prepared using the multifunctional additive, as compared to the corresponding control dispersions, suggests that the size of pigment particles was considerably smaller when a pigment dispersion contained the multifunctional additive. The smaller particle-size obtained with the use of the multifunctional additive is due to deflocculation of pigments, demonstrating the pigment dispersancy functionality of the multifunctional additive. The pigment dispersancy functionality of the multifunctional additive was also confirmed microscopically.

### Example 7

This example shows the composition (Table VIII) of a multifunctional additive in the form of an inverse emulsion, i.e., glycerin-in-oil emulsion, wherein the multi-functional additive comprises lauryl glucoside as the amphiphilic anchoring agent, which has an AAEF of greater than 1.65×10⁻³, as well as polyglyceryl-2 dipolyhydroxystearate as a second amphiphilic anchoring agent, which has an AAEF value of less than 1.65×10⁻³. An exemplary laboratory procedure for producing the inverted emulsion form of the multifunctional additive is described below, and it is noted that the procedure could possibly involve any mixing-shearing process known in the art, including extrusion.

TABLE VIII

| Phase | Ingredient | Weight % |
|---|---|---|
| A | Glyceryl Cassia Gum (Dry-basis) | 17 |
| | Glycerin | 34 |
| | Lauryl Glucoside (50 Wt. % Solution) | 4 |
| B | Polyglyceryl-2 Dipolyhydroxystearate | 11 |
| | Isostearyl Hydroxystearate | To 100% |

### Laboratory Procedure

i) Combine the Phase A ingredients in a suitable vessel and mix the resulting mixture under a relatively low speed of a mixer (e.g., kitchen-aid mixer) until homogeneous
ii) Combine the Phase B ingredients under vigorous mixing, after heating isostearyl hydroxystearate (a low-melting waxy material) to about 45° C. to melt the wax
iii) Under strong agitation, using a Caframo mixer fitted with a dispersion-blade agitator, add the Phase A mixture in small portions to the Phase B mixture
iv) Continue shearing the batch under a relatively high mixer-speed until homogeneous or lump-free

### Example 8

This example shows the method and composition for producing a multifunctional additive that is particularly useful in stabilizing complex oil-in-water (O-W) emulsions. As stated previously, by complex emulsion it is meant an O-W emulsion that contains both dispersed solid particles, such as pigment (e.g., Zinc Oxide) particles, and dispersed enhanced oil-droplets suspended in the water-phase of the emulsion. The composition of the multifunctional additive is given in Table IX, followed by a description of a method for producing the additive.

TABLE IX

| Phase | Ingredient | Weight % |
|---|---|---|
| A | Deionized Water | 65.455 |
| A | Multifunctional Additive from EXAMPLE 7 | 3.636 |
| B | Zinc Oxide (Z-cote, Supplier: BASF) | 30.909 |

### Procedure

i) Combine deionized water and the multifunctional additive (inverse emulsion) of EXAMPLE 7 in a suitable vessel
ii) Homogenize or shear the batch under a relatively high mixer-speed, using a Caframo mixer fitted with a dispersion-blade agitator, until the inverse emulsion inverts (breaks) completely and the resulting mixture shows an increased viscosity due to the dissolution of glyceryl cassia gum in the water-phase
iii) While continuing to homogenize the batch, add Zinc Oxide powder in small portions
iv) Continue to homogenize the batch until homogeneous or lump-free

### Example 9

This example shows the method and composition for producing a stable, complex oil-in-water (O-W) emulsion, using the multifunctional additive.

TABLE X

| Phase | Ingredient | Weight % |
|---|---|---|
| A | Deionized Water | 59.9010 |
| | Multifunctional Additive from EXAMPLE 7 | 3.9604 |
| B | Sunflower Oil | 17.4604 |
| C | Multifunctional Additive from EXAMPLE 8 | 17.9851 |
| D | 25.2% Solution of EDTA (Disodium Salt of Eth- | 0.6931 |
| | ylenediaminetetracetic acid), pH-adjusted to 8 | |
| | with Sodium Hydroxide | |

### Procedure

i) Combine the Phase A ingredients in a suitable vessel
ii) Homogenize or shear the mixture from (i) under a relatively high mixer-speed, using a Caframo mixer fitted with a dispersion-blade agitator, until the inverse emulsion inverts (breaks) completely and the resulting mixture shows an increased viscosity due to the dissolution of glyceryl cassia gum in the water-phase
iii) Add Phase B, and continue to homogenize the batch under a relatively high mixer-speed until uniform
iv) Add Phase C, and continue to homogenize the batch under a relatively high mixer-speed until uniform
v) While continuing to homogenize the batch, add Phase B and mix until uniform

The above complex emulsion tested stable (per the heating-centrifuging method described in EXAMPLE 3) against oil-separation as well as any heavy flocculation of the zinc oxide particles.

### Example 10

This example shows that a multifunctional additive comprising a mixture of glyceryl cassia gum (Component 1) and a particulate thickening agent (Component 2), smectite clay, produces a much higher low-shear-rate viscosity (Brookfield viscosity at 0.5 rpm of spindle speed) in aqueous compositions, as compared to each of the individual components of the additive. Table XI demonstrates this functional benefit, presenting some exemplary aqueous compositions along with their respective low-shear-rate viscosities, wherein the aqueous compositions were produced following the procedure described below.

TABLE XI

| | Low-shear- | Ingredient, Weight % | | |
|---|---|---|---|---|
| | rate Viscosity, | 2% (w/w) | 5% (w/w) | |
| | cps 0.5 rpm | Glyceryl | Smectite | |
| | spindle-speed, | Cassia Gum | Clay Sus- | |
| Aqueous | Brookfield | Solution in | pension in | Deion- |
| Composition | RVT | Deionized | Deionized | ized |
| Sample | Viscometer | Water | Water | Water |
| Sample 1: | 200 | 25.0 | | 75.0 |
| With Component | | | | |
| 1 of the | | | | |
| Multifunctional | | | | |
| Additive | | | | |
| Sample 2: | 2,400 | 50.0 | | 50.0 |
| With Component | | | | |
| 1 of the | | | | |
| Multifunctional | | | | |
| Additive | | | | |
| Sample 3: | 7,000 | | 40.0 | 60.0 |
| With Component | | | | |
| 2 of the | | | | |
| Multifunctional | | | | |
| Additive | | | | |
| Sample 4: | 61,800 | 25.0 | 40.0 | 35.0 |
| With the | | | | |
| Multifunctional | | | | |
| Additive | | | | |
| Sample 5: | 80,000 | 50.0 | 40.0 | 10.0 |
| With the | | | | |
| Multifunctional | | | | |
| Additive | | | | |

### Procedure (Samples 4 and 5)

i) Combine deionized water and the smectite clay suspension in a suitable vessel, and shear the suspension at a relatively high mixer-speed (1,000 rpm), using a Caframo mixer fitted with a dispersion-blade agitator
ii) Add the glyceryl cassia gum solution, and continue shearing the mixture at a relatively high mixer-speed (1,000-2,750 rpm) until uniform Samples 1 and 2 were prepared by mixing or diluting a 2% (w/w) solution of glyceryl cassia gum in deionized water. Sample 3 was prepared by shearing a 5% (w/w) suspension of smectite in deionized, using a Caframo mixer fitted with a dispersion-blade agitator, at a mixer-speed of 1,000-2,750 rpm.

As apparent from the viscosity-results presented in Table XII, the aforementioned multifunctional additive of the present invention produces synergistic thickening in aqueous compositions. The high-shear-rate viscosities (Brookfield RVT viscosity at 20 rpm) were also significantly higher for Samples 4 and 5, than for Samples 1-3, in Table XI.

### Example 11

This example shows that a multifunctional additive comprising a mixture of glyceryl cassia gum (Component 1) and a natural polymer-based thickening agent (Component 2), xanthan gum, produces a much higher low-shear-rate viscosity (Brookfield viscosity at 0.5 rpm of spindle speed) in aqueous compositions as compared to each of the individual components of the additive. Table XII demonstrates this functional benefit, presenting some exemplary aqueous compositions along with their respective low-shear-rate viscosities, wherein the aqueous compositions were produced following a procedure described below.

TABLE XII

| | Low-shear- | Ingredient, Weight % | | |
|---|---|---|---|---|
| | rate Viscosity, | 2% (w/w) | 2% (w/w) | |
| | cps 0.5 rpm | Glyceryl | Xanthan | |
| | spindle-speed, | Cassia Gum | Gum | |
| Aqueous | Brookfield | Solution in | Solution in | Deion- |
| Composition | RVT | Deionized | Deionized | ized |
| Sample | Viscometer | Water | Water | Water |
| Sample 1: | 11480 | | 30.0 | 70.0 |
| With Component | | | | |
| 2 of the | | | | |
| Multifunctional | | | | |
| Additive | | | | |
| Sample 2: | 51600 | 6.0 | 24.0 | 70.0 |
| With the | | | | |
| Multifunctional | | | | |
| Additive | | | | |
| Sample 3: | 98000 | 10.0 | 20.0 | 70.0 |
| With the | | | | |
| Multifunctional | | | | |
| Additive | | | | |
| Sample 4: | 103200 | 15.0 | 15.0 | 70.0 |
| With the | | | | |
| Multifunctional | | | | |
| Additive | | | | |
| Sample 5: | 136000 | 20.0 | 10.0 | 70.0 |
| With the | | | | |
| Multifunctional | | | | |
| Additive | | | | |
| Sample 6: | 110400 | 24.0 | 6.0 | 70.0 |
| With the | | | | |
| Multifunctional | | | | |
| Additive | | | | |
| Sample 7: | 70 | 30.0 | | 70.0 |
| With Component | | | | |
| 1 of the | | | | |
| Multifunctional | | | | |
| Additive | | | | |

The aqueous compositions presented in Table XII were prepared by mixing the ingredients under strong agitation, using a Caframo mixer fitted with a dispersion-blade agitator. The viscosity-results presented in Table XII show that the aforementioned multifunctional additive produces synergistic thickening in aqueous compositions.

### Example 12

This example shows that the synergistic thickening in aqueous compositions, due to a multifunctional additive of the type described in EXAMPLE 11 is possible even when an aqueous composition contains a relatively high amount (1.1% by weight) of an electrolyte, sodium chloride (NaCl). As shown in Table XIII, a multifunctional additive comprising a mixture of glyceryl cassia gum and xanthan gum produced synergistic thickening in an aqueous composition with a relatively high electrolyte-content, as long as the weight-ratio of glyceryl cassia gum to xanthan gum was below a certain level.

TABLE XIII

| | Low-shear- | Ingredient, Weight % | | |
|---|---|---|---|---|
| | rate Viscosity, | 2% (w/w) | 2% (w/w) | |
| Aqueous | cps 0.5 rpm | Glyceryl | Xanthan | |
| Composition | spindle-speed, | Cassia Gum | Gum | |
| Sample: Each | Brookfield | Solution in | Solution in | Deion- |
| with 1.1% by | RVT | Deionized | Deionized | ized |
| weight of NaCl | Viscometer | Water | Water | Water |
| Sample 1: | 48900 | | 30.0 | 68.9 |
| With Component | | | | |
| 2 of the | | | | |
| Multifunctional | | | | |
| Additive | | | | |
| Sample 2: | 70000 | 6.0 | 24.0 | 68.9 |
| With the | | | | |
| Multifunctional | | | | |
| Additive | | | | |
| Sample 3: | 82800 | 10.0 | 20.0 | 68.9 |
| With the | | | | |
| Multifunctional | | | | |
| Additive | | | | |
| Sample 4: | 52800 | 12.0 | 18.0 | 68.9 |
| With the | | | | |
| Multifunctional | | | | |
| Additive | | | | |
| Sample 5: | 42000 | 20.0 | 10.0 | 68.9 |
| With the | | | | |
| Multifunctional | | | | |
| Additive | | | | |
| Sample 6: | Too thin to | 30.0 | | 68.9 |
| With Component | measure | | | |
| 1 of the | | | | |
| Multifunctional | | | | |
| Additive | | | | |

Based on the data in examples 11 and 12, it is speculated that as the level of electrolyte in a composition is decreased, the effective weight ratio of galactomannan to xantham gum that provides thickening synergy will broaden and vice versa; as the electrolyte level increases, the effective weight ratio of galactomannan to xantham gum that provides thickening synergy will narrow. However, at standard levels of electrolyte for personal care and cosmetic compositions, it can be seen that a synergy exists between galactomannan and xantham gum over a fairly broad ratio.

### Example 13

This example shows the composition and method for a multifunctional additive in a dry powder form.

In a 3-L bowl of a kitchen-aid mixer was weighed 68.68 g of an aqueous solution (50 wt. %) of lauryl glucoside, an amphiphilic anchoring agent compound of the present invention. After turning on the mixer at a low speed, 51.2 g of glyceryl cassia solids was added to the lauryl glucoside solution. The mixture was left under slow agitation for about 45 minutes, following which it was dried in an oven to a volatile matter-content of about 4 wt. %. The dried mixture was pulverized into fine powder-like solids by shearing it through agitation in the kitchen-aid bowl mixer.

### Example 14

This example shows synergistic thickening in aqueous emulsion compositions, due to a multifunctional additive comprising a mixture of glyceryl cassia gum and a cross-linked polymer-based thickening agent, for example, the polymers known in the art as Carbomer™ or Carbopol™ polymers.

TABLE XIV

| | Weight % | | |
|---|---|---|---|
| | Emulsion 1 | | Emulsion 3 |
| | Dry Blend + | Emulsion 2 | Carbopol |
| | Carbopol | Dry Blend | 980 |
| Ingredient | 980 | Only | Only |
| Multifunctional Additive: | 1.00 | 1.00 | |
| Glyceryl Cassia (GC) Dry | | | |
| Blend of EXAMPLE 13 | | | |
| 57.5% GC + 38.5% Lauryl | | | |
| Glucoside | | | |
| Carbomer (Carbopol 980) | 0.35 | | 0.35 |
| Lauryl Glucoside | | | 0.385 |
| Sodium Chloride | 0.25 | 0.25 | 0.25 |
| Sunflower Oil | 20.00 | 20.00 | 20.00 |
| Water, Preservative, to | | | |
| 100% | | | |
| Viscosity, cps, 20 rpm, | 11,600 | 550 | 1,500 |
| RV | | | |

As may be noted from Table XIV, Emulsion 1, comprising a multifunctional additive and Carbomer, has a significantly higher viscosity than Emulsions 2 and 3 which do not contain either the multifunctional additive or Carbomer

These emulsions were produced using an emulsification method similar to the ones described in the previous examples, using a dispersion blade agitator and at an ambient temperature.

Each of the documents referred to above is incorporated herein by reference. The mention of any document is not an admission that such document qualifies as prior art or constitutes the general knowledge of the skilled person in any jurisdiction. Except in the Examples, or where otherwise explicitly indicated, all numerical quantities in this description specifying amounts of materials, reaction conditions, molecular weights, number of carbon atoms, and the like, are to be understood as modified by the word “about.” It is to be understood that the upper and lower amount, range, and ratio limits set forth herein may be independently combined. Similarly, the ranges and amounts for each element of the technology can be used together with ranges or amounts for any of the other elements. As used herein, the expression “consisting essentially of” permits the inclusion of substances that do not materially affect the basic and novel characteristics of the composition under consideration.

1. A multi-functional additive comprising:
a. a substrate having at least one hydrogen bonding site, said substrate comprising at least one of:
i. a solid particle, wherein said at least one hydrogen bonding site is on a surface of said solid particle, or
ii. an amphiphillic anchoring agent, said amphiphillic anchoring agent being comprised of (A) an oil soluble fatty tail, and (B) a polar portion that provides said at least one hydrogen bonding site, wherein the ratio (“R_{H/Mw}”) between the number of hydrogen bonding sites on the polar portion (B) to the weight average molecular weight, Mw, of the amphiphillic anchoring agent is between about 1.65×10⁻³ and 0.2,
b. glyceryl substituted cassia tora gum wherein the glycerol molar substitution is from 0.25:1 to 3:1 glycerol to cassia tora gum.

2. The multi-functional additive of claim 1 wherein said substrate comprises said solid particle.

3. The multi-functional additive of claim 1 wherein said substrate consists of said solid particle.

4. The multi-functional additive of claim 2, wherein the solid particle is an inorganic pigment.

5. The multi-functional additive of claim 2, wherein the solid particle is a hydrophilic particle.

6. The multi-functional additive of claim 2, wherein the solid particle is a hydrophilically modified particle.

7. The multi-functional additive of claim 1 wherein said substrate comprises said amphiphillic anchoring agent.

8. The multi-functional additive of claim 1 wherein said substrate consists of said amphiphillic anchoring agent.

9. The multi-functional additive of claim 7 wherein the amphiphillic anchoring agent is an amphiphillic fatty acid, amphiphillic fatty ester, amphiphillic fatty alcohol, amphiphillic phospholipid, amphiphillic sterol or amphiphillic polymer.

10. The multi-functional additive of claim 9, wherein the amphiphillic anchoring agent has an anchoring agent efficiency factor (AAEF) of greater than about 1.65×10⁻³.

11.-14. (canceled)

15. The multi-functional additive of claim 14 wherein the glycerol molar substitution is 1:1 glycerol to cassia tora gum.

16. The multi-functional additive of claim 6, wherein the weight ratio of the glycerol substituted cassia tora gum to the amphiphillic anchoring agent is 0.1 or greater.

17. The multi-functional additive of claim 1 wherein the composition additionally comprises a synergistic amount of a thickening agent.

18. The multi-functional additive of claim 17 wherein the thickening agent is a particulate thickener selected from the group consisting of smectite clay, fumed silica, fumed alumina, laponite, and mixtures thereof or a polymeric thickener selected from the group consisting of xanthan gum, acrylate cross-polymers, hydroxyethyl cellulose, and mixtures thereof.

19. The multi-functional additive of claim 1, additionally comprising a personal care or cosmetically acceptable hydrophilic medium.

20. The multi-functional additive of claim 1, additionally comprising a personal care or cosmetically acceptable hydrophobic medium.

21. The multi-functional additive of claim 20, wherein the hydrophobic medium is selected from waxes, oils, silicone fluids, and combinations thereof.

22. A personal care or cosmetically acceptable composition comprising:
a. a substrate having at least one hydrogen bonding site, said substrate comprising at least one of:
i. a solid particle, wherein said at least one hydrogen bonding site is on a surface of said solid particle,
ii. an amphiphillic anchoring agent, said amphiphillic anchoring agent being comprised of (A) an oil soluble fatty tail, and (B) a polar portion that provides said at least one hydrogen bonding site, wherein the ratio (“R_{H/Mw}”) between the number of hydrogen bonding sites on the polar portion (B) to the Mw of the amphiphillic anchoring agent is between about 1.65×10⁻³ and 0.2, or
iii. both (i) and (ii),
b. a glyceryl substituted cassia tora gum wherein the glycerol molar substitution is from 0.25:1 to 3:1 glycerol to cassia tora gum,
c. a personal care or cosmetically acceptable hydrophilic medium, and
d. a personal care or cosmetically acceptable hydrophobic medium,
wherein said water-soluble galactomannan polymer is bonded to said substrate.

23. The composition of claim 22, wherein the hydrophilic medium is selected from water, alchohols, glycols, polyols, glycerin, and combinations thereof.

24. The composition of claim 22, wherein the hydrophobic medium is selected from waxes, oils, silicone fluids, and combinations thereof.

25. A method of providing a colloidally stable dispersion of a hydrophobic medium in a continuous hydrophilic medium comprising including in the dispersion an amphiphillic anchoring agent and a glyceryl substituted cassia tora gum.

26. A method for dispersing both solid particles and hydrophobic media in a hydrophilic medium comprising selecting at least one solid particle and selecting at least one hydrophobic medium, and mixing the solid particle and at least one hydrophobic medium in the hydrophilic medium along with at least one amphiphillic anchoring agent and at least one glyceryl substituted cassia tora gum.

27. A method for dispersing solid particles in a hydrophilic medium comprising selecting at least one solid particle, and mixing the solid particle in the hydrophilic medium along with at least one glyceryl substituted cassia tora gum.
